(19) United States
(12) Patent Application Publication
(10) Pub. No.: US 2024/0139415 A1
(43) Pub. Date: May 2, 2024

(54) SYSTEM AND METHOD FOR DETECTING PRESSURE INDUCED SENSOR ATTENUATIONS (PISAs) OF CONTINUOUS GLUCOSE MONITORING (CGM)

(71) Applicant: UNIVERSITY OF VIRGINIA PATENT FOUNDATION, Charlottesville, VA (US)

(72) Inventors: Boris P. KOVATCHEV, Charlottesville, VA (US); Benjamin J. LOBO, Charlottesville, VA (US)

(73) Assignee: UNIVERSITY OF VIRGINIA PATENT FOUNDATION, Charlottesville, VA (US)

(21) Appl. No.: 18/500,212

(22) Filed: Nov. 2, 2023

Related U.S. Application Data
(60) Provisional application No. 63/421,931, filed on Nov. 2, 2022.

(51) Int. Cl. A61M 5/172; G16H 20/17

(52) U.S. Cl. CPC A61M 5/1723; G16H 20/17; A61M 2230/201

(57) ABSTRACT

Embodiments can relate to a system for automatically detecting sensor compression in continuous glucose monitoring including at least one sensor and at least one processor in communication with the at least one sensor, the at least one processor executing at least two machine learning models, wherein the at least one processor is programmed or configured to cause the processor to receive, from the at least one sensor, measurement data including at least one time series of blood glucose (BG) measurements measured by the at least one sensor, determine that the at least one time series of BG measurements is a candidate series including a compression artifact using a first machine learning model, and generate, using a second machine learning model, a signal output indicating that the at least one time series of BG measurements was obtained while the at least one sensor was subject to compression.

### CROSS-REFERENCE TO RELATED APPLICATIONS

This patent application is related to and claims the benefit of priority to U.S. provisional patent application No. 63/421,931, filed on Nov. 2, 2022, the entire contents of which is incorporated by reference.

### FIELD

An aspect of embodiments generally relates to medicine and medical devices, as used for monitoring of blood sugar levels in the treatment of diabetes mellitus and other metabolic disorders, including but not limited to type 1 and type 2 diabetes, type 2 (T1D, T2D), latent autoimmune diabetes in adults (LADA), postprandial or reactive hyperglycemia, or insulin resistance. Embodiments relate to systems and methods for detecting that a continuous glucose monitoring (CGM) sensor is subject to compression and for detection of pressure induced sensor attenuations (PISAs).

### BACKGROUND INFORMATION

Past advancements in continuous glucose monitoring (CGM) devices have contributed to the treatment of diabetes and led to some contemporary closed-loop control systems (e.g., artificial pancreas). Despite advances in continuous glucose monitoring (CGM), CGM sensors may be vulnerable to compression artifacts (e.g., Pressure Induced Sensor Attenuations (PISAs)). Compression artifacts may occur in measured data when a sensor is pressed (e.g., subject to compression) while collecting measurements. For example, compression artifacts may occur when a subject having a sensor attached sleeps on an area (e.g., arm) where the sensor is inserted. Compression artifacts may be characterized by a rapid drop in magnitude of sensor measurements (e.g., a low reading), followed by an eventual recovery of sensor measurements (e.g., a normal reading). Such drops in sensor measurements can result in false hypoglycemia alarms, insulin shutoff in low glucose suspend or closed-loop systems, and other effects negatively impacting treatment of diabetes. Reliable methods to detect and/or anticipate compression artifacts do not exist. Compression artifact prediction and thereby prevention methods have not been previously developed.

### SUMMARY

An exemplary embodiment can relate to a system for automatically detecting sensor compression in continuous glucose monitoring. The system may include at least one sensor. The system may include at least one processor in communication with the at least one sensor. The at least one processor may execute at least two machine learning models. The at least one processor may be programmed or configured to cause the processor to receive, from the at least one sensor, measurement data including at least one time series of blood glucose (BG) measurements measured by the at least one sensor. The at least one processor may be programmed or configured to cause the processor to determine that the at least one time series of BG measurements is a candidate series including a compression artifact using a first machine learning model. The at least one processor may be programmed or configured to cause the processor to generate, using a second machine learning model, a signal output indicating that the at least one time series of BG measurements was obtained while the at least one sensor was subject to compression.

An exemplary embodiment can relate to a system for automatically detecting onset of sensor compression in continuous glucose monitoring. The system may include at least one sensor. The system may include at least one processor in communication with the at least one sensor. The at least one processor may execute program code for at least one machine learning model. The at least one processor may be programmed or configured to cause the processor to receive, from the at least one sensor, measurement data including at least one time series of blood glucose (BG) measurements measured by the at least one sensor. The at least one processor may be programmed or configured to cause the processor to determine that the at least one time series of BG measurements is a candidate series including BG measurements representing onset of sensor compression. The at least one processor may be programmed or configured to cause the processor to input a time series sub-sequence of at least one time series of BG measurements into at least one machine learning model. The at least one processor may be programmed or configured to cause the processor to generate, using at least one machine learning model, a signal output indicating that at least one BG measurement was obtained while the at least one sensor was subject to compression.

An exemplary embodiment can relate to a computer-implemented method for generating at least one machine learning model to accurately detect sensor compression in continuous glucose monitoring. The method may include receiving, as an input to a processor, at least one training dataset. The at least one training dataset may include plural time series of blood glucose (BG) measurements. The method may include determining plural time series sub-sequences based on the training dataset. At least one time series sub-sequence may include at least one BG measurement value that is less than a compression estimate threshold. The method may include extracting one or more features from each of the plural time series sub-sequence. The method may include inputting the one or more features from the plural time series sub-sequences into at least one machine learning model for training. The method may include detecting a sensor compression based on providing at least one time series of BG measurement as input to the at least one machine learning model.

### BRIEF DESCRIPTION OF THE DRAWINGS

Other features and advantages of the present disclosure will become more apparent upon reading the following detailed description in conjunction with the accompanying drawings, wherein like elements are designated by like numerals, and wherein:

FIG. 1 shows an exemplary system configuration for an embodiment of a system for detecting sensor compression in continuous glucose monitoring as disclosed herein;

FIG. 2 shows an exemplary method for detecting sensor compression in continuous glucose monitoring as disclosed herein;

FIG. 3 shows an exemplary system implementation for detecting sensor compression in continuous glucose monitoring as disclosed herein;

FIG. 4A shows an exemplary plot of sensor measurements in continuous glucose monitoring with visualizations of sensor measurements including pressure induced sensor attenuations as disclosed herein;

FIG. 4B shows an exemplary plot of sensor measurements in continuous glucose monitoring with visualizations of sensor measurements without pressure induced sensor attenuations as disclosed herein;

FIG. 5 shows an exemplary distribution of pressure induced sensor attenuation durations for an exemplary training dataset as disclosed herein;

FIG. 6A shows an exemplary candidate series including a time series of blood glucose measurements including a time series sub-sequence as disclosed herein;

FIG. 6B shows an exemplary time series of blood glucose measurements including plural candidate series each including a time series sub-sequence as disclosed herein;

FIG. 7A shows an exemplary plot of a time series of blood glucose measurements including plural pressure induced sensor attenuations and plural drop time-windows as disclosed herein;

FIG. 7B shows an exemplary plot of a time series of blood glucose measurements including plural pressure induced sensor attenuations and plural drop time-windows as disclosed herein;

FIG. 8A shows an exemplary plot of a time series of blood glucose measurements including plural pressure induced sensor attenuations and plural drop time-windows having a predicted probability of pressure induced sensor attenuation onset as disclosed herein;

FIG. 8B shows an exemplary plot of a time series of blood glucose measurements including plural pressure induced sensor attenuations and plural drop time-windows each having a predicted probability of pressure induced sensor attenuation onset as disclosed herein;

FIG. 9 shows an exemplary plot of area under the receiver operating characteristics curve and a precision-recall curve for classifier models used to classify time series of blood glucose measurements as including an onset of a pressure induced sensor attenuation to detect sensor compression as disclosed herein;

FIG. 10A shows an exemplary system configuration of an exemplary computing device as disclosed herein;

FIG. 10B shows an exemplary system environment in which systems, methods, and/or computer-readable media may be implemented as disclosed herein;

FIG. 11 is a block diagram of an exemplary computing system in which systems, methods, and/or computer-readable media may be implemented as disclosed herein;

FIG. 12 shows an exemplary environment in which systems, methods, and/or computer-readable media may be implemented as disclosed herein; and

FIG. 13 is a block diagram of an exemplary machine in which systems, methods, and/or computer-readable media may be implemented as disclosed herein.

### DETAILED DESCRIPTION

Various embodiments provide for the ability to detect continuous glucose monitoring (CGM) sensor compression lows (e.g., compression artifacts) in real time and thereby prevent compression low effects negatively impacting the treatment of diabetes. Such negative impacts that may be prevented by various embodiments include false hypoglycemia alarms, and insulin shutoff in insulin delivery systems. Various embodiments may fully identify pressure induced sensor attenuations (PISAs) in a prospective manner to provide an indication that a PISA is occurring or is about to occur in real time. Embodiments may improve accuracy of CGM sensors by detecting compression artifacts inherent with CGM devices. Embodiments improve action of continuous subcutaneous insulin infusion therapy and related systems, such as sensor-augmented pump (SAP), low glucose suspend (LGS), predictive low glucose suspend (PLGS), or automated insulin delivery (AID) (e.g., an artificial pancreas).

Various embodiments improve functioning of a computer (e.g., computer processor) to automatically detect PISAs and improve the treatment of diabetes. A computer that is not programmed or configured with aspects of some embodiments could not automatically detect PISAs in CGM sensor and diabetes treatment prior to the development of various embodiments disclosed herein. According to embodiments, CGM sensors may no longer be vulnerable to compression artifacts. Compression artifacts may be automatically detected in real time in measured data when a sensor is pressed (e.g., subject to compression) while collecting measurements. For example, embodiments may detect compression artifacts when a subject having a sensor attached sleeps on an area (e.g., arm) where the sensor is inserted. Embodiments may detect both a rapid drop in magnitude of sensor measurements (e.g., a drop time-window), and an eventual recovery of sensor measurements (e.g., a rise time-window). Embodiments may detect PISAs in such sensor measurements to anticipate and/or prevent false hypoglycemia alarms, insulin shutoff in low glucose suspend or closed-loop systems, and other effects that may negatively impact treatment of diabetes. Embodiments may accurately predict compression artifacts such that compression artifacts can be handled in real time when they occur to enhance treatment of diabetes.

Various embodiments use machine learning models and techniques to analyze large amounts of data. The data analyzed and used to build machine learning models of the various embodiments may be vast and complex. For example, embodiments may use one or more signals provided by CGM sensors, which may include, but are not limited to: raw blood glucose estimate (prior to calibration and temperature corrections), filtered blood glucose, temperature, time of day, time of sensor life, and/or the like. Such signals and data may be supplied to a machine learning model and/or technique as disclosed herein. Embodiments may use additional information and/or signals from other PISA detection techniques, considerations regarding glycemic events such as hypoglycemia, or data from various anticipated future sensors, such as a pressure sensor included with a CGM sensor. As an example, embodiments may use CGM data traces from hundreds of CGM sensors containing tens of thousands of hours of sensor data including thousands of PISA events.

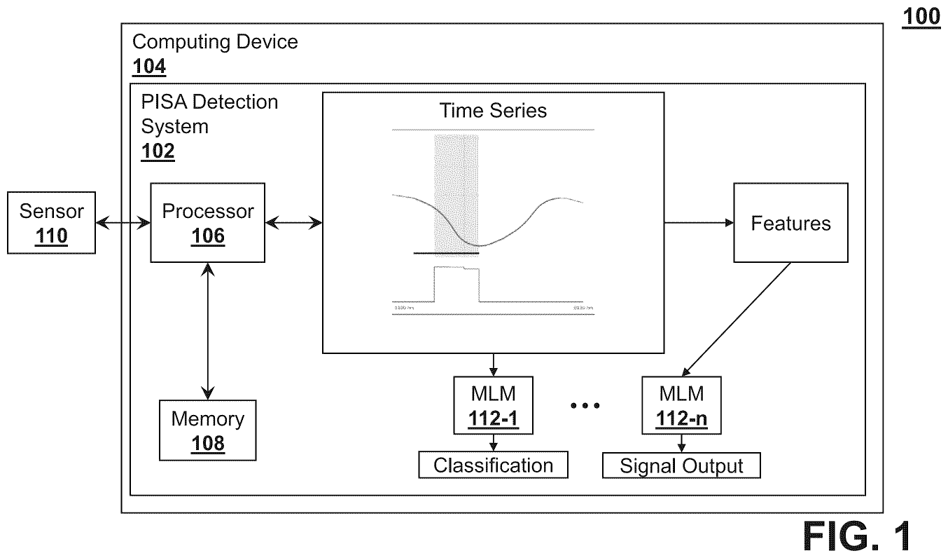

FIG. 1 shows an exemplary system configuration 100 for an embodiment of a system operable via program code (e.g., software instructions executed by a processor) for detecting sensor compression in CGM. The various components of FIG. 1 may be implemented in and/or processed by a processor (e.g., a central processing unit (CPU)) and/or on any number of distributed processors (e.g., a distributed computing system) coupled with memory and connected via a communications network. Each of the components shown in FIG. 1 are described in the context of an exemplary embodiment.

As shown in FIG. 1, embodiments relate to a system configured for detecting sensor compression in CGM. In some embodiments, system configuration 100 may automatically detect sensor compression in CGM in real time. In some embodiments, system configuration 100 may include pressure induced sensor attenuation (PISA) detection system 102, computing device 104, processor 106, memory 108, sensor 110, and machine learning models (MLMs) 112-1 through 112-n (referred to individually as MLM 112 and collectively as plural MLMs 112 where appropriate).

In some embodiments, system configuration 100 may include at least one sensor (e.g., sensor 110). The at least one sensor may measure and/or collect BG measurements associated with time stamps. The BG measurements (e.g., BG measurement data) may be in the form of a time series including plural time stamps. For example, each BG measurement measured and/or collected by the at least one sensor may be associated with exactly one time stamp, such that a collection of plural BG measurements forms a time series of BG measurements collected by the at least one sensor over time. The at least one time series of BG measurements may span various amounts of time and may be of any various lengths (e.g., number of time stamps and BG measurements in a time series). For example, at least one time series of BG measurements may include 30 minutes (e.g., in duration) of measurement data measured by at least one sensor. The at least one sensor may measure and/or collect BG measurements over time at specified time intervals (e.g., every 30 seconds, every minute, and/or the like) which may define a resolution of a time series of BG measurements. For example, plural time stamps of a time series of BG measurements may be separated by any one or more of 30 second intervals, 1 minute intervals, 2.5 minute intervals, and/or 5 minute intervals. Other time intervals may be used for collecting measurements and/or for resolution of time series.

In some embodiments, system configuration 100 may include at least one processor (e.g., processor 106) in communication with the at least one sensor. The at least one processor may execute program code for at least two machine learning models (e.g., MLMs 112). In some embodiments, at least one processor may execute the at least two machine learning models concurrently. The at least one processor may execute program code for detecting sensor compression in CGM.

In some embodiments, at least one processor may be programmed or configured (e.g., via software instructions) to cause the processor to receive, from the at least one sensor, measurement data including at least one time series of BG measurements measured by the at least one sensor. The measurement data may be transmitted from the at least one sensor to the at least one processor in real time (e.g., with respect to the sensor collecting the measurement data). Alternatively, the measurement data may be transmitted from the at least one sensor to memory (e.g., memory 108) coupled with the at least one processor such that the at least one processor may access the measurement data at a later time with respect to when the at least one sensor collected the measurement data.

In some embodiments, the at least one time series of BG measurements may include plural time stamps. Each time stamp may be associated with a BG measurement. For example, each time stamp may represent a relative or absolute time for when the BG measurement was collected by a sensor (e.g., sensor 110). In some embodiments, the plural time stamps may be separated by any one or more of 30 second intervals, 1 minute intervals, 2.5 minute intervals, and/or 5 minute intervals.

In some embodiments, at least one processor may be programmed or configured to cause the processor to determine that the at least one time series of BG measurements is a candidate series including a compression artifact using a first machine learning model. A candidate series may include a time series of BG measurements that includes a drop time-window (e.g., a series of BG measurements decreasing in value over time). A candidate series may include a time series of BG measurements that includes one or more BG measurement values below a BG measurement threshold (e.g., a threshold value). In some embodiments, a candidate series may include a time series of BG measurements that contains one or more attributes that may indicate that the time series of BG measurements includes a compression artifact. However, in some instances, a candidate series may contain one or more attributes indicating that the time series of BG measurements includes a compression artifact, while the time series of BG measurements does not include a compression artifact. In other instances, the candidate series will include a compression artifact. Determining that the at least one time series of BG measurements is a candidate series may be a first step for finding a compression artifact in measurement data and detecting sensor compression.

In some embodiments, at least one processor may be programmed or configured to cause the processor to determine that the at least one time series of BG measurements includes a change in BG measurement values across plural time stamps (e.g., across two consecutive time stamps, or across multiple time stamps beginning from a first time stamp). The change in BG measurement values may exceed a threshold (e.g., a threshold value). For example, at least one processor may calculate a change in BG measurement values by determining a difference between a first BG measurement value and a second BG measurement value to determine the change in BG measurement values. At least one processor may then determine that the difference between the first BG measurement value and the second BG measurement value exceeds a threshold (e.g., a drop time-threshold, a rise time-threshold, and/or the like). In some embodiments, the first BG measurement value may be associated with a first time stamp and the second BG measurement value may be associated with a second time stamp, where the first time stamp and second time stamp are consecutive time stamps in the at least one time series of BG measurements. Alternatively, the first BG measurement value may be associated with a first time stamp and the second BG measurement value may be associated with a second time stamp, where the first time stamp and second time stamp are separated by one or more time stamps in the at least one time series of BG measurements.

In some embodiments, the at least one processor, as configured to determine that the at least one time series of BG measurements is a candidate series, may be programmed or configured to cause the processor to determine that the at least one time series of BG measurements includes a time series sub-sequence having a drop time-window and having a rise time-window associated with the drop time-window. A time series sub-sequence may include a series of BG measurements associated with time stamps in the form of a time series including plural time stamps that is a subset of the at least one time series of BG measurements. For example, each BG measurement measured and/or collected by the at least one sensor may be associated with exactly one time stamp, such that a collection of plural BG measurements forms a time series of BG measurements collected by the at least one sensor over time. The time series of BG measurements may include one or more time series sub-sequences that may span various amounts of time and may be of any various lengths (e.g., number of time stamps and BG measurements in a time series sub-sequence) within the time series of BG measurements.

In some embodiments, the at least one processor may determine (e.g., by identifying plural time stamps in at least one time series of BG measurements) the time series sub-sequence using at least one machine learning model. In some embodiments, the time series sub-sequence may include a sequence of time stamps corresponding to at least a portion of the drop time-window (e.g., at least one time stamp of the time series sub-sequence is within the drop time-window) and at least a portion of the rise time-window (e.g., at least one time stamp of the time series sub-sequence is within the rise time-window). In some embodiments, the time series sub-sequence may span at least 2.5 minutes in duration of BG measurements.

In some embodiments, at least one processor, as configured to determine that the at least one time series of BG measurements is a candidate series, may be programmed or configured to cause the processor to determine a drop time-window within the at least one time series based on a difference between a first BG measurement value and a second BG measurement value exceeding a drop time-threshold. The drop time-window may include a series of plural time stamps and plural BG measurements that begin at a first time stamp associated with the first BG measurement (e.g., BG measurement value) and end at a second time stamp associated with the second BG measurement (e.g., BG measurement value). In some embodiments, the first BG measurement value and the second BG measurement value may be part of the same measurement data (e.g., time series of BG measurements).

In some embodiments, at least one processor, as configured to determine that the at least one time series of BG measurements is a candidate series, may be programmed or configured to cause the processor to determine a rise time-window within the at least one time series based on a difference between a third BG measurement value and a fourth BG measurement value exceeding a rise time-threshold. The rise time-window may include a series of plural time stamps and plural BG measurements that begin at a third time stamp associated with the third BG measurement (e.g., BG measurement value) and end at a fourth time stamp associated with the fourth BG measurement (e.g., BG measurement value). A rise time-window may be associated with a drop time-window in that a rise time-window can only occur within a time series of BG measurements after at least one drop time-window has occurred.

In some embodiments, a drop-time threshold may be equal to 10 mg/dL (e.g., a BG measurement value). In some embodiments, a rise-time threshold may be equal to 6 mg/dL (e.g., a BG measurement value).

In some embodiments, at least one processor, as configured to determine that the at least one time series of BG measurements is a candidate series, may be programmed or configured to cause the processor to determine a rolling mean for the at least one time series of BG measurements. A rolling mean may include one or more new values corresponding to the BG measurements in the time series, including a smooth BG value associated with each BG measurement and time stamp pair.

In some embodiments, at least one processor, as configured to determine that the at least one time series of BG measurements is a candidate series, may be programmed or configured to calculate an indicator value for the smooth BG value at each time stamp t, wherein the indicator value is equivalent to a Boolean true where:

BGₜ−BG_{t-lag}>BG threshold

where t is a current time stamp for which an indicator value is determined, BGₜ is the smooth BG value at time stamp t, BG_{t-lag} is a smooth BG value at a previous time stamp, lag is a measure of time such that t-lag represents the previous time stamp, and BG threshold represents a BG threshold value. In some embodiments, a difference between a first smooth BG value associated with the first time stamp and a second smooth BG value associated with the second time stamp is greater than 7.5 mg/dL (e.g., a BG measurement value).

In some embodiments, the lag may be equivalent to 5 minutes and the BG threshold may be equivalent to 10.0 mg/dL (e.g., a BG measurement value).

In some embodiments, at least one processor, as configured to determine that the at least one time series of BG measurements is a candidate series, may be programmed or configured to identify a time series sub-sequence in the at least one time series of BG measurements wherein the time series sub-sequence has a set of indicator values, the set of indicator values beginning at a first time stamp and ending at a second time stamp.

In some embodiments, at least one processor may be programmed or configured to cause the processor to identify, with at least one machine learning model, the rise time-window associated with the drop time-window as occurring within a range of 15 minutes to 180 minutes later than the drop time-window in the at least one time series. For example, a rise time-window may be identified as beginning at a rise start time stamp (e.g., a time stamp at the beginning of a rise time-window) that occurs 40 minutes after a drop end time stamp (e.g., a time stamp at the end of a drop time-window).

In some embodiments, at least one processor may be programmed or configured to cause the processor to input a time series sub-sequence of at least one time series of BG measurements into at least one machine learning model. For example, at least one processor may be programmed or configured to cause the processor to input the time series sub-sequence (e.g., the time stamps and/or BG measurement values associated with the time stamps) into at least one machine learning model for training, testing, and/or for generating a prediction and/or signal output. Additionally or alternatively, at least one processor may be programmed or configured to cause the processor to input one or more features of the time series sub-sequence (e.g., other data associated with the time series sub-sequence that may be extracted from the time series sub-sequence, such as time of day, type of sensor, and/or the like) into at least one machine learning model for training, testing, and/or for generating a prediction and/or a signal output.

In some embodiments, at least one processor may be programmed or configured to cause the processor to generate, using a second machine learning model, a signal output indicating that the at least one time series of BG measurements was obtained while the at least one sensor was subject to compression. For example, the signal output may include a signal transmitted to an insulin delivery system to cause the insulin delivery system to perform an action, a signal transmitted to a display, a signal transmitted to another processor to cause the processor to perform an action, and/or the like.

In some embodiments, at least one processor, as configured to generate a signal output, may be programmed or configured to cause the processor to predict that the at least one sensor is subject to compression while the at least one sensor is obtaining a BG measurement. At least one processor may be programmed or configured to cause the processor to predict (e.g., generate a prediction) via at least one machine learning model executed by the processor. The processor may generate a prediction in real time via outputting an indication based on receiving measurement data from the at least one sensor as the sensor is collecting the measurement data (e.g., in real time with respect to the sensor collecting the measurement data).

In some embodiments, at least one processor, as configured to generate a signal output, may be programmed or configured to cause the processor to predict, in real time via outputting an indication, that the at least one sensor is subject to compression while the at least one sensor is obtaining a BG measurement. For example, at least one processor may generate a prediction based on a probability value generated by at least one machine learning model. The probability value may represent a probability that a time series sub-sequence includes a BG measurement obtained while the at least one sensor was subject to compression. In this way, at least one machine learning model may be used to generate a probability that a time series sub-sequence includes a BG measurement obtained while the at least one sensor was subject to compression, and that probability value may represent a measure of confidence for the machine learning model in predicting that the at least one sensor is subject to compression while the at least one sensor is obtaining a BG measurement (e.g., a BG measurement that is included in the time series sub-sequence analyzed by the at least one machine learning model). A higher probability may represent a higher likelihood that the BG measurements in the time series sub-sequence were collected by the at least one sensor while the at least one sensor was subject to compression.

In some embodiments, at least one processor is programmed or configured to cause the processor to determine a maximum probability value of plural probability values generated by at least one machine learning model. The plural probability values may be associated with plural time stamps in the time series sub-sequence that are contained within a drop time-window. For example, each probability value of the plural probability values may be associated with at least one time stamp in the at least one time series of BG measurements. That is, a probability value may represent a probability that the BG measurement associated with a time stamp in the time series sub-sequence was obtained while the at least one sensor was subject to compression.

In some embodiments, at least one processor is programmed or configured to cause the processor to determine the probability that the time series sub-sequence includes a BG measurement obtained while the at least one sensor was subject to compression based on the maximum probability value. For example, a maximum probability value may be determined out of the plural probability values, where each probability value is associated with a time stamp and a BG measurement. In some other embodiments, an average probability value, or another probability and/or statistical metric may be used.

In some embodiments, at least one processor may be programmed or configured to cause the processor to identify one or more features of the at least one time series of BG measurements. For example, the at least one processor may identify and/or extract features of the at least one time series of BG measurements for inputting into at least one machine learning model (e.g., via feature extraction techniques). At least one processor may be programmed or configured to cause the processor to input the one or more features into at least one machine learning model (e.g., the second machine learning model) for classification and/or generating a signal output based on a classification of the at least one time series of BG measurements.

In some embodiments, at least one processor may be in combination with an insulin delivery system that is in communication with the at least one processor. The at least one processor may be programmed or configured to cause the processor to transmit a signal output to the insulin delivery system. The signal output may indicate that the at least one sensor is subject to compression, and the signal output may cause the insulin delivery system to perform at least one or more of initiating insulin delivery (e.g., after insulin delivery was stopped due to compression artifacts), continuing insulin delivery (e.g., after determining that the at least one sensor is subject to compression and is functioning correctly), disabling an alarm (e.g., after sensor compression caused triggering of an alarm), and/or any combination thereof.

Referring to FIG. 1, PISA detection system 102 may include software instructions (e.g., program code) implemented on computing device 104. PISA detection system 102 may include memory 108 storing the software instructions. PISA detection system 102 may include processor 106 executing the software instructions to cause processor 106 to perform one or more functions. PISA detection system 102 may include sensor 110 (e.g., a CGM sensor).

PISA detection system 102 may include at least two machine learning models that are trained with BG measurement data (e.g., MLM 112-1 to MLM 112-n). At least one machine learning model may generate at least one signal output based on at least one time series of BG measurement data having a compression artifact supplied as a runtime input to at least one machine learning model. A signal output of at least one machine learning model may include a prediction (e.g., a determination) whether the at least one time series of BG measurement data was obtained while the at least one sensor was subject to compression. The at least two machine learning models may be trained using one or more time series of BG measurements received by computing device 104 and/or processor 106 from memory 108 and/or sensor 110. Additionally or alternatively, at least one machine learning model may generate at least one signal output (e.g., prediction) based on training datasets, testing datasets and/or other datasets.

In some embodiments, PISA detection system 102 may be implemented in a single computing device. In some embodiments, PISA detection system 102 may be implemented in plural computing devices (e.g., a group of servers, such as a group of computing devices 104, and/or the like) as a distributed system such that software instructions and/or machine learning models are implemented on different computing devices. In some embodiments, PISA detection system 102 may be associated with computing device 104, such that PISA detection system 102 is executed on computing device 104 or a portion of PISA detection system 102 is executed on computing device 104 as part of a distributed computing system where sensor 110 is not part of computing device 104. Alternatively, PISA detection system 102 may include at least one computing device 104 executing software instructions and at least one sensor 110 for detecting PISAs.

Sensor 110 may include a CGM sensor that is configured to detect and/or measure blood glucose within a subject (e.g., a patient). In some embodiments, sensor 110 may include one or more sensors. For example, sensor 110 may include a CGM sensor and a pressure sensor. In some embodiments, sensor 110 may include plural CGM sensors. Sensor 110 may be worn and/or attached to a subject on various parts of the subject's body (e.g., arm, abdomen, or the like). Sensor 110 may be configured to collect measurements (e.g., BG measurements) and transmit measurements to a processor (e.g., processor 106). Sensor 110 may be subject to compression (e.g., via a subject, or other means) while sensor 110 is collecting and/or obtaining measurements, thereby affecting an accuracy of the measurements obtained by sensor 110.

Sensor 110 may include a sampling rate that may be configured for collecting and/or obtaining measurements over time having a particular sampling resolution. For example, sensor 110 may be configured to sample measurements at 30 second intervals, 1 minute intervals, 2.5 minute intervals, 5 minute intervals, and/or the like. Sensor 110 may transmit sampled measurements to processor 106 in real time as measurements are sampled. In some embodiments, sensor 110 may transmit at least one time series of sampled measurements to processor 106 and/or memory 108 at specified time intervals. A time series may include plural BG measurements, each BG measurement associated with a time stamp. The time stamp may represent an absolute or relative time when the BG measurement was collected by sensor 110. Sensor 110 may be in communication with computing device 104 and/or processor 106 via wired (e.g., a data bus, ethernet, and/or the like) or wireless (e.g., Wi-Fi, Bluetooth, and/or the like) means and/or a communication interface.

Computing device 104 may include processor 106 (e.g., CPU) and memory 108. Processor 106 may execute software instructions (e.g., compiled program code) for PISA detection system 102, including software instructions for at least two machine learning models (e.g., trained machine learning models). In some embodiments, sensor 110 may be separate from computing device 104. Alternatively, sensor 110 may be integrated with (e.g., part of) computing device 104.

Computing device 104 may include one or more processors (e.g., processor 106) configured to execute software instructions. For example, computing device 104 may include a desktop computer, a portable computer (e.g., laptop computer, tablet computer), a workstation, a mobile device (e.g., smartphone, cellular phone, personal digital assistant, wearable device), a server, and/or other like devices. Computing device 104 may include a computing device configured to communicate with one or more other computing devices over a network. Computing device 104 may include a group of computing devices (e.g., a group of servers) and/or other like devices. In some embodiments, computing device 104 may include a data storage device. Alternatively, a data storage device may be separate from computing device 104 and may be in communication with computing device 104.

Processor 106 may be implemented in hardware, software, or a combination of hardware and software. For example, processor 106 may include a common processor (e.g., a CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed with software instructions such that the processor is configured to cause the processor to perform functions when executing the software instructions. In some embodiments, processor 106 may include plural processors implemented in a single computing device 104 (e.g., a CPU and a GPU) or processor 106 may include plural processors implemented among plural distributed computing devices 104. Processor 106 may be coupled to memory 108 via a data bus to transfer data between processor 106 and memory 108. In some embodiments, processor 106 may be coupled to sensor 110 via wired (e.g., a data bus, ethernet, and/or the like) or wireless (e.g., Wi-Fi, Bluetooth, and/or the like) means and/or communication interface.

Memory 108 may include random access memory (RAM), read-only memory (ROM), and/or another type of dynamic or static storage device (e.g., flash memory, magnetic memory, optical memory, etc.) that stores information and/or software instructions for use by processor 106. Memory 108 may include a computer-readable medium and/or storage component. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 108 from another computer-readable medium or from another device via a communication interface with computing device 104. When executed, software instructions stored in memory 108 and executed by processor 106 may cause processor 106 to perform one or more functions described herein. Embodiments described herein are not limited to any specific combination of hardware circuitry and software.

MLMs 112 may include at least two machine learning models (e.g., MLM 112-1, MLM 112-2, etc.). MLMs 112 may be trained using unsupervised and/or supervised training methods. MLMs 112 may be trained with a training dataset received from a data storage device. At least one MLM 112 of plural MLMs 112 may generate a first signal output (e.g., a prediction) based on a runtime input provided to at least one MLM 112 (e.g., a trained MLM 112). The first signal output by may be provided as input to another MLM 112 to generate an additional signal output based on the first signal output. In some embodiments, at least one MLM 112 may generate a first signal output (e.g., a prediction classifying a runtime input; a classification) with a first MLM 112-1 based on a runtime input. The first signal output (e.g., the classification) may then be an input (e.g., as a feature vector) to a second MLM 112-2 such that MLM 112-2 generates a second and/or final signal output. In some embodiments, the first signal output may be provided as input to a second MLM 112-2 based on certain criteria (e.g., based on a certain classification). Various input/output patterns may be used by MLMs 112 for any number and/or arrangement of MLMs 112s to generate a final signal output. In this way, MLMs 112 may use various structures and/or input/output patterns of MLMs 112 to generate signal outputs and/or predictions (e.g., classifications, probabilities, and/or the like). In some embodiments, PISA detection system 102 (e.g., via processor 106) may execute MLMs 112 concurrently.

The number and arrangement of systems, hardware, and/or modules (e.g., software instructions) shown in FIG. 1 is provided as an example. There may be additional systems, hardware, and/or modules, fewer systems, hardware, and/or modules, different systems, hardware, and/or modules, or differently arranged systems, hardware, and/or modules than those shown in FIG. 1. Furthermore, two or more systems, hardware, and/or modules shown in FIG. 1 may be implemented within a single system, hardware, and/or module. A single system, hardware, and/or module shown in FIG. 1 may be implemented as multiple, distributed systems, hardware, and/or modules. Additionally or alternatively, a set of systems, a set of hardware, and/or a set of modules (e.g., one or more systems, one or more hardware devices, one or more modules) of FIG. 1 may perform one or more functions described as being performed by another set of systems, another set of hardware, or another set of modules of FIG. 1.

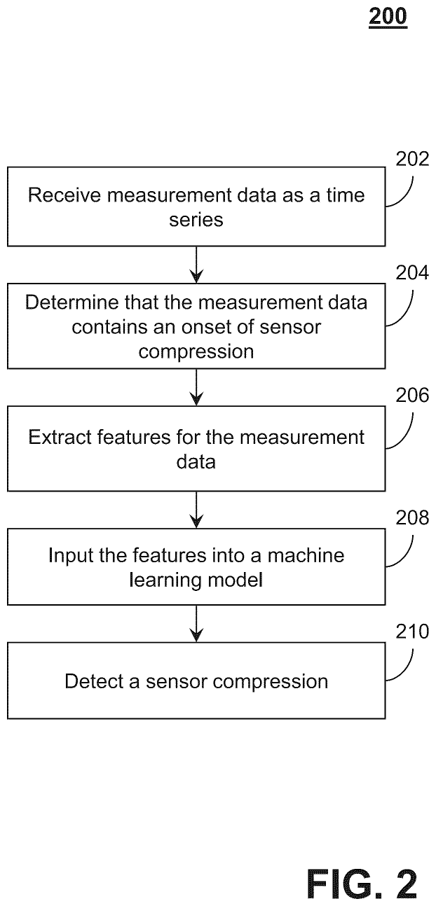

As shown in FIG. 2, embodiments relate to an exemplary method 200 for detecting sensor compression in continuous glucose monitoring as disclosed herein. Method 200 may be performed by one or more components of system configuration 100. In some embodiments, one or more of the functions described with respect to method 200 may be performed (e.g., completely, partially, etc.) by PISA detection system 102 (e.g., via processor 106). In some embodiments, one or more of the steps of method 200 may be performed (e.g., completely, partially, etc.) by another system, hardware, or module or a group of systems, hardware, or modules separate from or including PISA detection system 102, such as a client device and/or a separate computing device.

In some embodiments, one or more of the steps of method 200 may be performed in a training phase for at least one MLM 112. A training phase for at least one MLM 112 may include a computing environment where a machine learning model (e.g., MLM 112-1) is being trained (e.g., a training environment, a model building phase, and/or the like). In some embodiments, one or more of the steps of method 200 may be performed in a testing phase for at least one MLM 112. A testing phase for at least one MLM 112 may include a computing environment where a machine learning model (e.g., MLM 112-1) is being tested and/or evaluated (e.g., a testing environment, model evaluation, model validation, and/or the like). In some embodiments, one or more of the steps of method 200 may be performed in a runtime phase for at least one MLM 112. A runtime phase for at least one MLM 112 may include a computing environment where a machine learning model (e.g., MLM 112-1) is active (e.g., deployed, accessible to client devices as a service, etc.) and is capable of generating signal outputs (e.g., runtime predictions) based on runtime inputs.

As shown in FIG. 2, at step 202, method 200 may include receiving measurement data as at least one time series. For example, PISA detection system 102 (e.g., via computing device 104 and/or processor 106) may receive at least one training dataset from sensor 110. In some embodiments, PISA detection system 102 may receive the measurement data for providing the measurement data as input to at least one MLM 112 for generating a classification and/or a signal output. The measurement data may include training data to train and/or test at least one MLM 112. Alternatively, the measurement data may include runtime inputs for at least one MLM 112. The at least one training dataset may include at least one time series of BG measurements.

In some embodiments, the measurement data may include plural time series of BG measurements. A time series of BG measurements may include plural time stamps. Each time stamp may be associated with a BG measurement. For example, one BG measurement collected by sensor 110 may be associated with exactly one time stamp. A time stamp may represent an absolute or relative time when the BG measurement was collected by sensor 110. Processor 106 may receive the measurements data including a time series of BG measurements as input to processor 106. A time series of BG measurements may span various amounts of time and may be of any various lengths (e.g., number of time stamps and BG measurements). For example, at least one time series of BG measurements may include at least 30 minutes of measurement data measured by at least one sensor. In this example, the number of time stamps and BG measurements may depend on the resolution of the BG measurements, or a sample rate at which sensor 110 collects the BG measurements. In some embodiments, a time series sub-sequence may span at least 2.5 minutes in duration of BG measurements. In some embodiments, a time series sub-sequence may be longer or shorter than a 2.5 minute duration.

At step 204, method 200 may include determining that the measurement data contains an onset of sensor compression. For example, PISA detection system 102 (e.g., via computing device 104 and/or processor 106) may determine that the measurement data received from sensor 110 includes at least one time series sub-sequences. In some embodiments, the measurement data may include plural time series sub-sequences. A time series sub-sequence may include at least one BG measurement value that is less than a compression estimate threshold. In some embodiments, the compression estimate threshold may be equivalent to 85 mg/dL (e.g., a BG measurement value).

In some embodiments, PISA detection system 102 may determine that the measurement data contains an onset of sensor compression based on determining that at least one BG measurement value is less than a compression estimate threshold. An onset of sensor compression may be represented in the measurement data as a compression artifact. For example, one or more BG measurements (e.g., within a time series of BG measurements) may form a compression artifact in the measurement data. The one or more BG measurements may indicate an onset of sensor compression (e.g., that sensor 110 was subject to compression while collecting the one or more BG measurements).

In some embodiments, PISA detection system 102 may determine that the measurement data received from sensor 110 contains an onset of sensor compression based on determining that the measurement data (e.g., a time series sub-sequence of the measurement data) contains at least one compression artifact. A compression artifact may include a time series of BG measurements where each BG measurement in the time series is below a compression estimate threshold such that the BG measurements are out of a normal range (e.g., outside of a band of equilibrium BG values indicating a normal BG level in a subject).

In some embodiments, PISA detection system 102 may determine that the measurement data contains an onset of sensor compression based on determining that the measurement data includes at least one drop time-window. A drop time-window may include a time series of BG measurements where the BG measurements decrease from an initial BG measurement value to a lower BG measurement value over the time series such that the BG measurement values cross a threshold BG measurement value that is between the initial BG measurement value and the lower BG measurement value. In some embodiments, a drop time-window may be defined by a time series of BG measurements where a difference between an initial BG measurement and a lower BG measurement exceeds a compression threshold. The initial BG measurement value and the lower BG measurement value may be separated in time (e.g., in the time series) by any number of time stamps (e.g., any amount of time) and are not required to be particularly separated in time.

In some embodiments, PISA detection system 102 may determine that the measurement data contains an end of sensor compression based on determining that the measurement data includes at least one rise time-window. A rise time-window may include a time series of BG measurements where the BG measurements increase from an initial BG measurement value to a higher BG measurement value over the time series such that the BG measurement values cross a threshold BG measurement value that is between the initial BG measurement value and the higher BG measurement value. In some embodiments, a rise time-window may be defined by a time series of BG measurements where a difference between an initial BG measurement and a higher BG measurement exceeds a compression threshold. The initial BG measurement value and the higher BG measurement value may be separated in time (e.g., in the time series) by any number of time stamps (e.g., any amount of time) and are not required to be particularly separated in time.

In some embodiments, PISA detection system 102 (e.g., via computing device 104 and/or processor 106) as configured to determine that the at least one time series of BG measurements is a candidate series, may determine a drop time-window within the at least one time series based on a difference between a first BG measurement and a second BG measurement exceeding a drop time-threshold. The drop time-window may include a series of plural time stamps and plural BG measurements that begin at a first time stamp associated with the first BG measurement and end at a second time stamp associated with the second BG measurement.

In some embodiments, PISA detection system 102 (e.g., via computing device 104 and/or processor 106), as configured to determine that the at least one time series of BG measurements is a candidate series, may determine a rise time-window within the at least one time series based on a difference between a third BG measurement and a fourth BG measurement exceeding a rise time-threshold. The rise time-window may include a series of plural time stamps and plural BG measurements that begin at a third time stamp associated with the third BG measurement and end at a fourth time stamp associated with the fourth BG measurement. A rise time-window may be associated with a drop time-window in that a rise time-window can only occur within a time series of BG measurements after at least one drop time-window has occurred.

In some embodiments, PISA detection system 102 may determine a drop time-threshold and/or a rise time-threshold. Alternatively, PISA detection system 102 may receive a value for a drop time-threshold and/or a value for a rise time-threshold as an input, for example, from a user of PISA detection system 102 (via one or more input devices and/or interfaces of computing device 104). In some embodiments, the drop-time threshold may be equal to 10 mg/dL and the rise-time threshold may be equal to 6 mg/dL, with respect to BG measurement values that me be calculated and/or determined based on BG measurements.

At step 206, method 200 may extract features for the measurement data. For example, PISA detection system 102 (e.g., via computing device 104 and/or processor 106) may extract one or more features from at least one time series of BG measurements of the measurement data based on determining the measurement data (e.g., at least one time series of the measurement data) contains one or more BG measurements indicating an onset of sensor compression. In some embodiments, PISA detection system 102 may extract the one or more features from a time series sub-sequence of the at least one time series of BG measurements. In some embodiments, PISA detection system 102 may provide the one or more features as input to at least one MLM 112 for training, testing, and/or to generate a signal output at runtime.

A time series sub-sequence may include a shorter time series of BG measurements with associated time stamps within the time series of BG measurements of the measurement data. For example, a first time series of BG measurements may be identified based on a predetermined time period used for analysis (e.g., a previous 30 minutes of BG measurement collected by sensor 110) while a time series sub-sequence may be identified as a shorter time series of BG measurements within the first time series of BG measurements (e.g., a 2.5 minute series of BG measurements within the previous 30 minutes of BG measurements.

In some embodiments, at least one processor, as configured to determine that the at least one time series of BG measurements is a candidate series, may be programmed or configured to cause the processor to determine a rolling mean for the at least one time series of BG measurements. A rolling mean may include one or more new values corresponding to the BG measurements in the time series, including a smooth BG value associated with each BG measurement and time stamp pair.

In some embodiments, PISA detection system 102 (e.g., via computing device 104 and/or processor 106), as configured to determine that the at least one time series of BG measurements is a candidate series, may calculate an indicator value for the smooth BG value at each time stamp t, wherein the indicator value is equivalent to a Boolean true where:

BGₜ-BG_{t-lag}>BG threshold

where t is a current time stamp for which an indicator value is determined, BGₜ is the smooth BG value at time stamp t, BG_{t-lag} is a smooth BG value at a previous time stamp, lag is a measure of time such that t-lag represents the previous time stamp, and BG threshold represents a BG threshold value. In some embodiments, PISA detection system 102 may determine a difference between a first smooth BG value associated with the first time stamp and a second smooth BG value associated with the second time stamp, where the difference is greater than 7.5 mg/dL.

In some embodiments, the lag may be equivalent to 5 minutes and the BG threshold may be equivalent to 10.0 mg/dL (e.g., a BG measurement value).

In some embodiments, PISA detection system 102 (e.g., via computing device 104 and/or processor 106), as configured to determine that the at least one time series of BG measurements is a candidate series, may be programmed or configured to identify a time series sub-sequence in the at least one time series of BG measurements wherein the time series sub-sequence has a set of indicator values, the set of indicator values beginning at a first time stamp and ending at a second time stamp.

In some embodiments, PISA detection system 102 (e.g., via computing device 104 and/or processor 106) may identify one or more features of the at least one time series of BG measurements. PISA detection system 102 may input the one or more features into at least one machine learning model (e.g., a second machine learning model) for classification and/or for generating a signal output.

At step 208, method 200 may input the features into a machine learning model. For example, PISA detection system 102 (e.g., via computing device 104 and/or processor 106) may input the one or more features from the at least one time series of BG measurements into at least one MLM 112 for training. In some embodiments, PISA detection system 102 may input the one or more features from at least one time series sub-sequence within the at least one time series of BG measurements into at least one MLM 112 for training. In some embodiments, PISA detection system 102 may input one or more features of a time series of BG measurements into at least one MLM 112 for testing and/or for generating a signal output at runtime.

At step 210, method 200 may detect a sensor compression. For example, PISA detection system 102 (e.g., via computing device 104 and/or processor 106) may detect a sensor compression based on providing at least one time series of BG measurements as input to the at least one machine learning model. In some embodiments, PISA detection system 102 may detect a sensor compression based on determining that at least one time series of BG measurements is a candidate series including a compression artifact using a first machine learning model (e.g., MLM 112-1). PISA detection system 102 may then determine that the candidate series includes a drop time-window of BG measurements. PISA detection system 102 may provide the candidate series (e.g., one or more features of the candidate series) as input to a second machine learning model (e.g., MLM 112-2).

PISA detection system 102 may generate, using the second machine learning model (e.g., MLM 112-2), a signal output indicating that the at least one time series of BG measurements (e.g., the candidate series) was obtained while the at least one sensor (e.g., sensor 110) was subject to compression. In some embodiments, PISA detection system 102 may generate the signal output based on determining a probability value representing a probability that the candidate series corresponds to an onset of sensor compression.

In some embodiments, PISA detection system 102 (e.g., via computing device 104 and/or processor 106), as configured to generate a signal output, may predict, in real time via outputting an indication, that the at least one sensor is subject to compression while the at least one sensor is obtaining a BG measurement. For example, PISA detection system 102 may generate a prediction based on a probability value generated by at least one machine learning model. The probability value may represent a probability that a time series sub-sequence includes a BG measurement obtained while the at least one sensor was subject to compression. In this way, PISA detection system 102 may use at least one machine learning model to generate a probability that a time series sub-sequence includes a BG measurement obtained while the at least one sensor was subject to compression, and that probability value may represent a measure of confidence for the machine learning model in predicting that the at least one sensor is subject to compression while the at least one sensor is obtaining a BG measurement (e.g., a BG measurement that is included in the time series sub-sequence analyzed by the at least one machine learning model). A higher probability may represent a higher likelihood that the BG measurements in the time series sub-sequence were collected by the at least one sensor while the at least one sensor was subject to compression.

In some embodiments, when PISA detection system 102 determines the probability value, PISA detection system 102 may generate a classification of the candidate series (e.g., via MLM 112-2) as “not subject to sensor compression” where the probability is lower than a probability threshold, or PISA detection system 102 may classify the candidate series (e.g., via MLM 112-2) as “subject to sensor compression” where the probability value is equal to or greater than the probability threshold. PISA detection system 102 may generate the signal output indicating that the at least one time series of BG measurements (e.g., the candidate series) was obtained while the at least one sensor was subject to compression based on PISA detection system 102 classifying the candidate series as “subject to compression.”

In some embodiments, PISA detection system 102 (e.g., via computing device 104 and/or processor 106) may determine the probability that the time series sub-sequence includes a BG measurement obtained while the at least one sensor was subject to compression based on a maximum probability value. For example, the maximum probability value may be determined out of plural probability values, where each probability value of the plural probability values is associated with at least one time stamp and at least one BG measurement in the time series sub-sequence. In some other embodiments, an average probability value, or another probability and/or statistical metric may be used by PISA detection system 102.

In some embodiments, PISA detection system 102 (e.g., via computing device 104 and/or processor 106) may determine that the at least one time series of BG measurements includes a change in BG measurements across plural time stamps (e.g., across two consecutive time stamps, or across multiple time stamps from a first time stamp). The change in BG measurements may exceed a threshold. For example, at least one processor may calculate a change in BG measurements by determining a difference between a first BG measurement value and a second BG measurement value to determine the change in BG measurements. At least one processor may then determine that the difference between the first BG measurement value and the second BG measurement value exceeds a threshold (e.g., a drop time-threshold, and/or the like).

In some embodiments, PISA detection system 102 (e.g., via computing device 104 and/or processor 106), as configured to determine that the at least one time series of BG measurements is a candidate series, may determine that the at least one time series of BG measurements includes a time series sub-sequence having a drop time-window and having a rise time-window associated with the drop time-window. In some embodiments, PISA detection system 102 may determine (e.g., by identifying plural time stamps in at least one time series of BG measurements) the time series sub-sequence using at least one machine learning model. In some embodiments, the time series sub-sequence may include a sequence of time stamps corresponding to at least a portion of the drop time-window (e.g., at least one time stamp of the time series sub-sequence is within the drop time-window) and at least a portion of the rise time-window (e.g., at least one time stamp of the time series sub-sequence is within the rise time-window). PISA detection system 102 may determine the time series sub-sequence based on determining that the time series sub-sequence includes a drop time-window and a corresponding rise time-window. The presence of the drop time-window and the corresponding rise time-window may be determined based on inputting the at least one time series of BG measurements into at least one machine learning model. In some embodiments, PISA detection system 102 may determine a drop time-window and a corresponding rise time-window based on determining a difference between a first BG measurement value and a second BG measurement value to determine a change in BG measurements (e.g., where the change in BG measurements occurs over time stamps in the drop time-window and/or time stamps in the rise time-window).

In some embodiments, PISA detection system 102 (e.g., via computing device 104 and/or processor 106) may predict that sensor 110 is subject to compression while sensor 110 is obtaining at least one BG measurement. For example, PISA detection system 102 may generate a prediction via MLM 112 executed by processor 106 based on inputting at least one time series of BG measurements (e.g., features thereof) to MLM 112. PISA detection system 102 may generate a prediction in real time via outputting an indication based on receiving measurement data (e.g., a time series of BG measurements) from sensor 110 as sensor 110 is collecting the measurement data (e.g., in real time with respect to sensor 110 collecting the measurement data and transmitting the measurement data to processor 106 and/or memory 108).

In some embodiments, the classification of the candidate series generated by PISA detection system 102 may include a prediction generated by a trained machine learning model (e.g., MLM 112-2) based on a runtime input provided to the trained machine learning model. The runtime input may include measurement data including at least one time series of BG measurements collected by sensor 110 in real time that is provided to a first machine learning model (e.g., MLM 112-1) to determine if the at least one time series of BG measurements contains a candidate series. In some embodiments, a candidate series may include a time series of BG measurements and/or a time series sub-sequence of BG measurements. PISA detection system 102 may then provide the candidate series to a second machine learning model (e.g., MLM 112-2) to generate a classification of the candidate series such that PISA detection system 102 may generate a signal output indicating that the at least one time series of BG measurements collected by sensor 110 in real time includes BG measurements that were collected by sensor 110 while sensor 110 was subject to compression. In this way, PISA detection system 102 may collect measurement data via sensor 110, provide the measurement data to at least two machine learning models, and generate a signal output indicating that sensor 110 is subject to compression in real time with respect to when the measurement data is collected by sensor 110.

In some embodiments, real time may include an instant in time with respect to the occurrence of an event (e.g., real time with respect to collection of measurement data) where a response (e.g., generating a signal output) may occur within a specified time, generally a relatively short time (e.g., within seconds or less) of the event occurring. For example, real time may refer to an instant in time where a signal output is generated by PISA detection system 102 concurrent with or shortly after (e.g., within milliseconds or seconds) the collection of measurement data by sensor 110. As a further example, a real-time (e.g., runtime) signal output may be generated with respect to inputting a time series of BG measurements to at least one machine learning model concurrent with or shortly after PISA detection system 102 receives the time series of BG measurements and/or concurrent with or shortly after PISA detection system 102 inputs the time series of BG measurements into at least one machine learning model for generating a runtime prediction and/or signal output.

In some embodiments, PISA detection system 102 (e.g., computing device 104 and/or processor 106) may be in combination with an insulin delivery system. PISA detection system 102 (e.g., via processor 106) may be in communication with the insulin delivery system (e.g., via wired and/or wireless means). PISA detection system 102 may transmit a signal output to the insulin delivery system, where the signal output indicates that sensor 110 is subject to compression while collecting BG measurements. The insulin delivery system may receive the signal output, where the signal output causes the insulin delivery system to perform at least one or more of initiating insulin delivery, continuing insulin delivery, disabling an alarm, and/or any combination thereof.

In some embodiments, PISA detection system 102 may label each of the plural time series sub-sequences with an indication that the time series sub-sequence includes a compression artifact or that the time series sub-sequence does not include a compression artifact. Labeling may be performed on one or more time series of BG measurements in a training and/or testing dataset.

Steps of method 200 may be performed in various orders and sequences and are not limited to being performed in the order shown in FIG. 2. For example, features may be extracted from training data and provided to a machine learning model for training prior to PISA detection system 102 receiving measurement from sensor 110. Similarly, in some instances, PISA detection system 102 may receive measurement data from sensor 110 following the detection of sensor compression. Accordingly, steps of method 200 are not limited to any particular order and may be performed on various components, whether implemented on a single computing device or multiple, distributed computing devices.

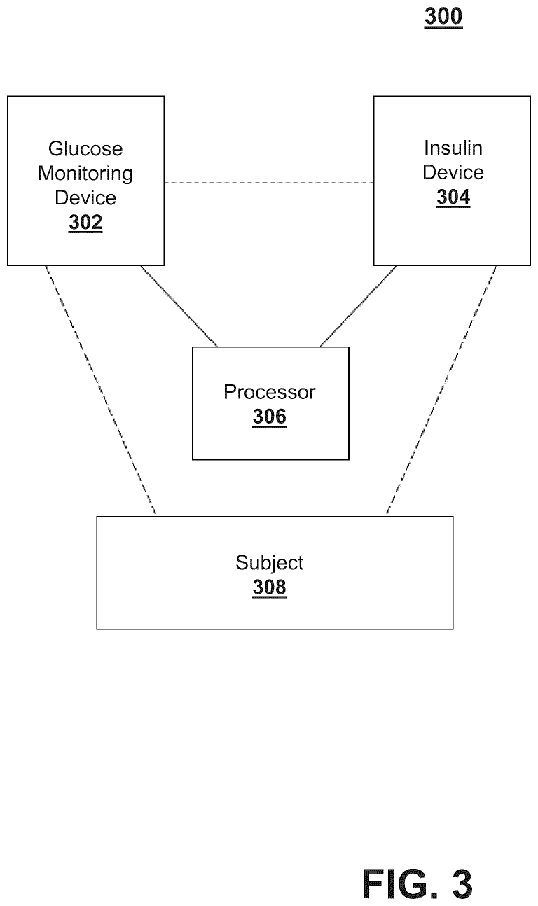

As shown in FIG. 3, embodiments may relate to an exemplary system implementation 300 for detecting sensor compression in continuous glucose monitoring. System 300 may include glucose monitoring device 302, insulin device 304, processor 306, and subject 308. In some embodiments, system 300 may include glucose monitoring device 302, processor 306, and subject 308, without insulin device 304. For example, system 300 may include glucose monitoring device 302 (e.g., and at least one sensor included therewith) in communication with at least one processor 306 to perform embodiments as disclosed herein.

In some embodiments, glucose monitoring device 302 and/or insulin device 304 may be the same as or similar to sensor 110. In some embodiments, glucose monitoring device 302 and/or insulin device 304 may include sensor 110. For example, glucose monitoring device 302 may include sensor 110 as a component of glucose monitoring device 302 or insulin device 304 may include sensor 110 as a component of insulin device 304. In some embodiments, glucose monitoring device 302, insulin device 304, and/or sensor 110 may be implemented in a single system. Alternatively, processor 306 may be implemented on a computing device separate from glucose monitoring device 302 and/or insulin device 304.

In some embodiments, glucose monitoring device 302 and/or insulin device 304 may include processor 306. For example, glucose monitoring device 302 may include processor 306 as a component of glucose monitoring device 302 or insulin device 304 may include processor 306 as a component of insulin device 304. In some embodiments, glucose monitoring device 302, insulin device 304, processor 306, and/or sensor 110 may be implemented in a single system. Alternatively, processor 306 may be implemented on a computing device separate from glucose monitoring device 302, insulin device 304, and/or sensor 110. Processor 306 may be implemented locally in glucose monitoring device 302, insulin device 304, or in a standalone device (e.g., computing device 104) (or in any combination of two or more of glucose monitoring device 302, insulin device 302, or a standalone device). In some embodiments, processor 306 may be the same as or similar to processor 106.

Glucose monitoring device 302 may include a device that may be used to monitor and/or test blood glucose levels of subject 308 (e.g., as a standalone device). Glucose monitoring device 302 may be affixed and/or attached to subject 308 to monitor blood glucose levels. Glucose monitoring device 302 may communicate (e.g., via a sensor, such as sensor 110) with subject 308 to monitor blood glucose levels of subject 308. In this way, glucose monitoring device 302 may collect measurement data (e.g., BG measurement data) for transmitting to processor 306 for detecting whether glucose monitoring device 302 and/or sensor 110 are subject to compression by subject 308. Processor 306 may execute software instructions (e.g., PISA detection system 102) as a component of glucose monitoring device 302 or separate from glucose monitoring device 302. For example, processor 306 may be implemented locally in glucose monitoring device 302. In some embodiments, glucose monitoring device 302 and insulin device 304 may be implemented each as a separate device or glucose monitoring device 302 and insulin device 304 may be implemented as a single device.

In some embodiments, glucose monitoring device 302 may generate outputs, errors, parameters for accuracy improvements, and/or accuracy related information that may be transmitted, such as to processor 306, for performing various analyses, such as error analyses and/or further improvements to embodiments herein.

Insulin device 304 may include an insulin delivery system, such as an insulin pump. Insulin device 304 may communicate with subject 308 to deliver insulin to subject 308. In some embodiments, processor 306 may execute software instructions (e.g., PISA detection system 102) as a component of insulin device 304 or separate from insulin device 304. For example, processor 306 may be implemented locally in insulin device 304. In some embodiments, insulin device 304 may be affixed and/or attached to subject 308 such that insulin device 304 may deliver insulin to the subject 308. Processor 306 and/or a portion of system 300 may be located remotely such that glucose monitoring device 302 and/or insulin device 304 may be operated as a telemedicine device.

Processor 306 may be implemented in hardware, software, or a combination of hardware and software. For example, processor 306 may include a common processor (e.g., a CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed with software instructions such that the processor is configured to cause the processor to perform functions when executing the software instructions. In some embodiments, processor 306 may include plural processors implemented in a single computing device (e.g., a CPU and a GPU) or processor 306 may include plural processors implemented among plural distributed computing devices. Processor 306 may be coupled to memory via a data bus to transfer data between processor 306 and memory. In some embodiments, processor 306 may be coupled to a sensor (e.g., sensor 110), glucose monitoring device 302 and/or insulin device 304 via wired (e.g., a data bus, ethernet, and/or the like) or wireless (e.g., Wi-Fi, Bluetooth, and/or the like) means and/or communication interface.

Subject 308 may include a patient located at their home or another desired location. In some embodiments, a subject may include a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

The number and arrangement of systems, hardware, and/or devices shown in FIG. 3 is provided as an example. There may be additional systems, hardware, and/or devices, fewer systems, hardware, and/or devices, different systems, hardware, and/or devices, or differently arranged systems, hardware, and/or devices than those shown in FIG. 3. Furthermore, two or more systems, hardware, and/or devices shown in FIG. 3 may be implemented within a single system, hardware, and/or device. A single system, hardware, and/or device shown in FIG. 3 may be implemented as multiple, distributed systems, hardware, and/or devices. Additionally or alternatively, a set of systems, a set of hardware, and/or a set of devices (e.g., one or more systems, one or more hardware components, one or more devices) of FIG. 3 may perform one or more functions described as being performed by another set of systems, another set of hardware, or another set of devices of FIG. 3.

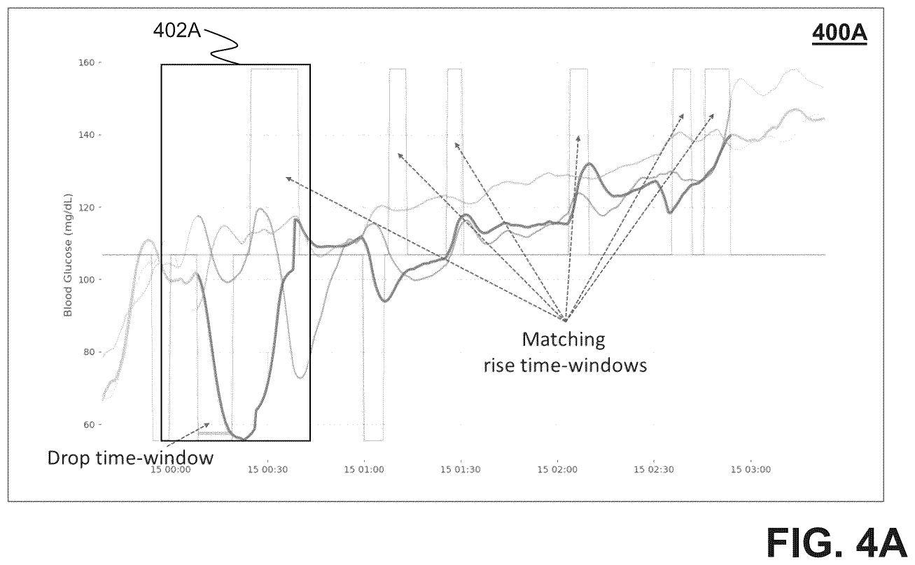

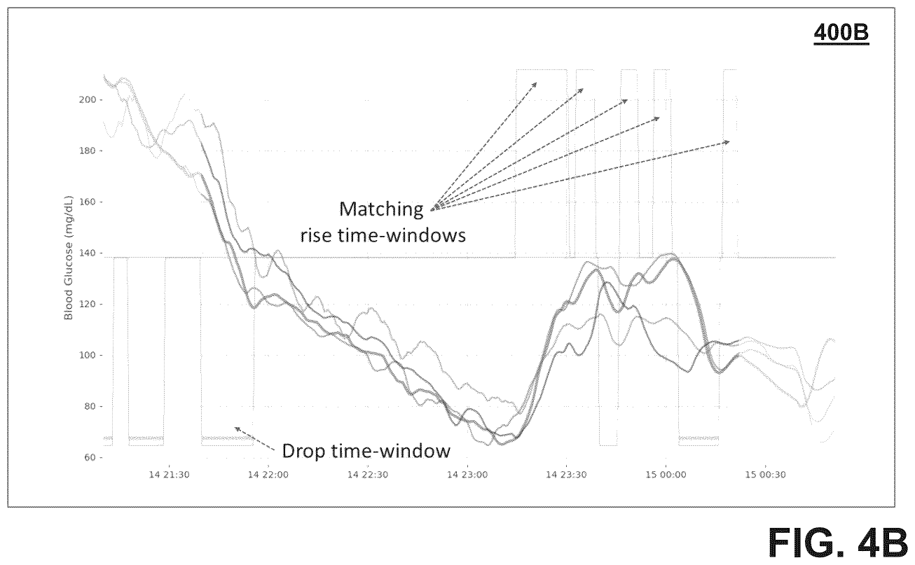

FIG. 4A shows an exemplary plot of sensor measurements (e.g., BG measurements collected by sensor 110) in continuous glucose monitoring with visualizations of sensor measurements including PISAs. For example, FIG. 4A shows examples of compression artifacts present in sensor measurements, where a sensor was subject to compression while collecting BG measurements. FIG. 4A shows a time series sub-sequence 402 which begins at a start of a drop time-window and ends at a rise end of a first rise time-window. Time series sub-sequence 402 may represent a PISA. That is, the BG measurements in time series sub-sequence 402 were collected by a sensor (e.g., sensor 110) that was subject to compression.

FIG. 4B shows an exemplary plot of sensor measurements (e.g., BG measurements collected by sensor 110) in continuous glucose monitoring with visualizations of sensor measurements without PISAs. For example, FIG. 4B shows no combination of a drop time-window and a rise time-window representing a PISA (e.g., compression artifact) for the sensor under consideration. The BG measurements shown in FIG. 4B (e.g., any of the time series sub-sequences) would not cause a processor (e.g., processor 106 and/or processor 306) to generate a signal output indicating that at least one time series of BG measurements was obtained while the sensor (e.g., sensor 110) that collected the BG measurement shown in FIG. 4B was subject to compression. For example, BG measurements shown in FIG. 4B were collected by a sensor that was not subject to compression.

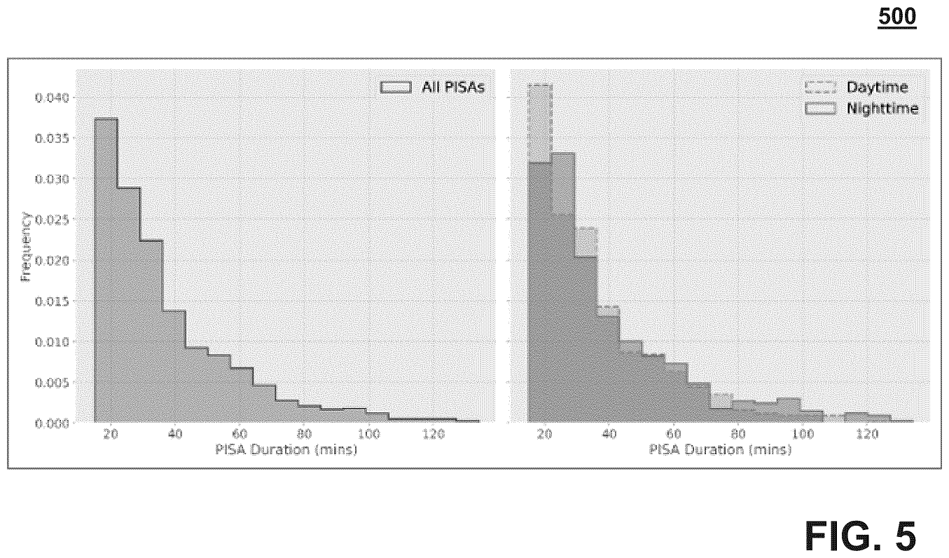

FIG. 5 is a histogram showing an exemplary distribution of PISA durations for an exemplary training dataset collected from one or more sensors. The left side of FIG. 5 shows an exemplary distribution of PISA durations for plural PISAs annotated in a training data set. The right side of FIG. 5 shows a same distribution based on a time of day when a PISA was collected by a sensor. FIG. 5 shows that training data may follow an exponential distribution with plural PISAs being less than 60 minutes in length. In some embodiments, there may be a slight difference in distribution of the length of a PISA based on when the PISA started. Based on FIG. 5, a duration of a PISA, a time of day of a PISA, and a duration of a PISA correlated with a time of day of a PISA may each be extracted as a feature from BG measurement data for input to at least one machine learning model (e.g., for training, testing, and/or runtime).

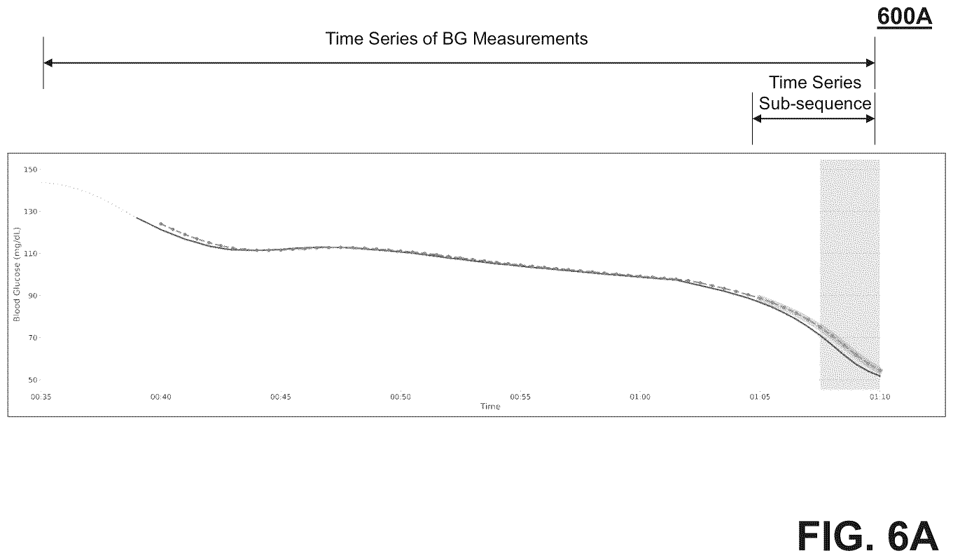

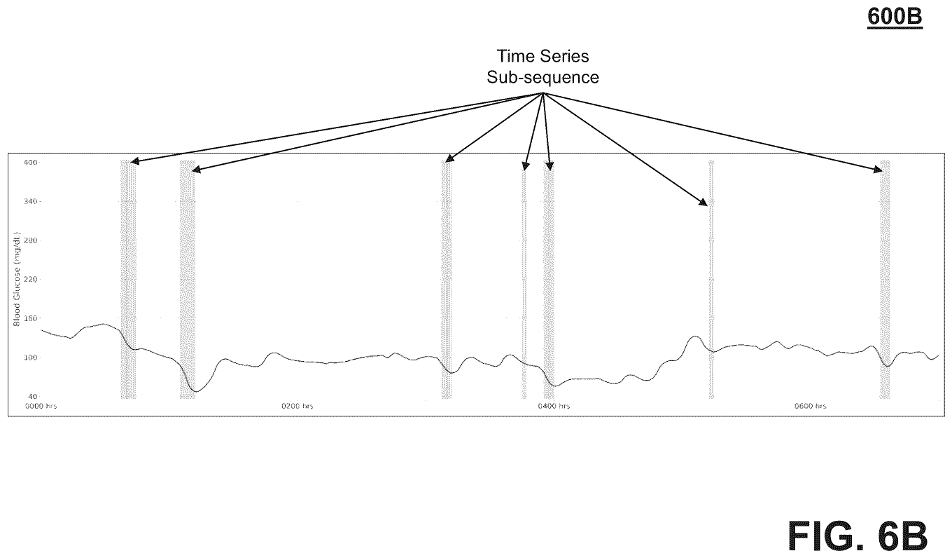

FIG. 6A shows a plot of exemplary BG measurements over time (e.g., a time series) including an exemplary candidate series including at least one time series of BG measurements. The at least one time series of BG measurements shown in FIG. 6A includes at least one time series sub-sequence. As shown in FIG. 6A, the at least one time series of BG measurements may include a time series of BG measurements collected by a sensor (e.g., sensor 110) over a time span (e.g., 30 minutes, and/or the like). The dashed line with circle markers shown in FIG. 6A may include a rolling mean that is calculated for the at least one BG time series. The at lest one time series of BG measurements may include at least one time series sub-sequence that is use to determine whether the at least one time series of BG measurements is a candidate series. The at least one time series of BG measurements may be of various lengths including various numbers of BG measurements and is not limited by the time series shown in FIG. 6A.

FIG. 6B shows a plot of an exemplary time series of BG measurements including plural candidate series each including a time series sub-sequence. FIG. 6B shows plural time series sub-sequences of the time series of BG measurements where each time series sub-sequence identified in FIG. 6B is identified as a candidate series (e.g., by PISA detection system 102).

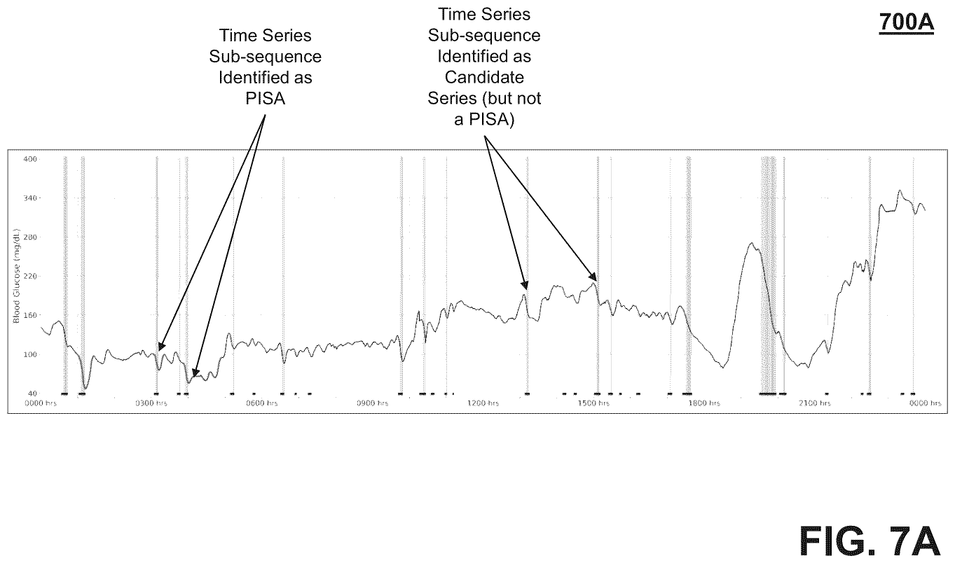

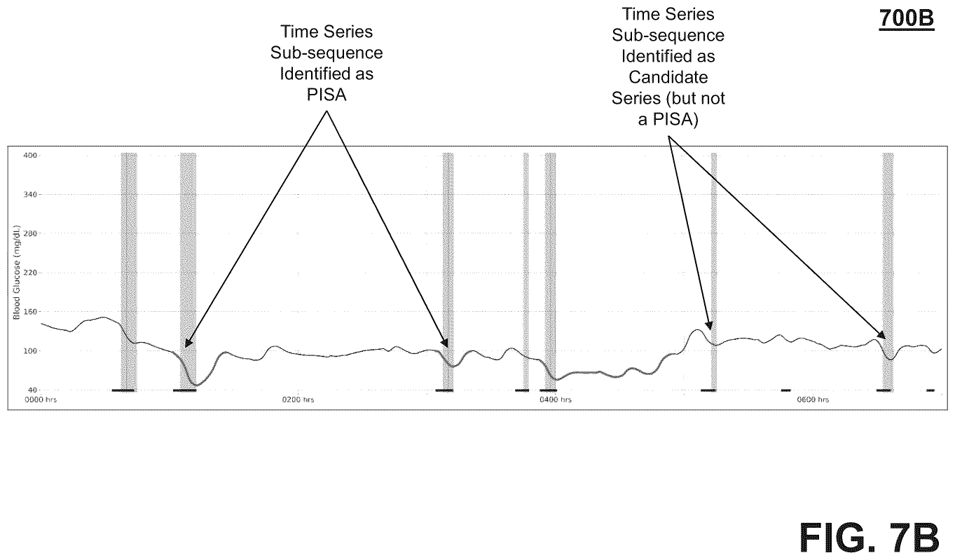

FIG. 7A shows an exemplary plot of a time series of BG measurements including plural PISAs and plural drop time-windows. For example, the time series sub-sequences identified in FIG. 7A as PISAs may have been determined to include a drop time-window after being determined to be a candidate series of BG measurements. In some embodiments, a portion of the candidate series in a time series of BG measurements may be identified as a PISA while some candidate series in a time series of BG measurements may be determined to include a drop time-window, but may not be determined to include a PISA.

FIG. 7B shows an exemplary plot of a time series of BG measurements including plural PISAs and plural drop time-windows as shown in FIG. 7A. FIG. 7B shows a portion of the time series of BG measurements that are shown in FIG. 7A.

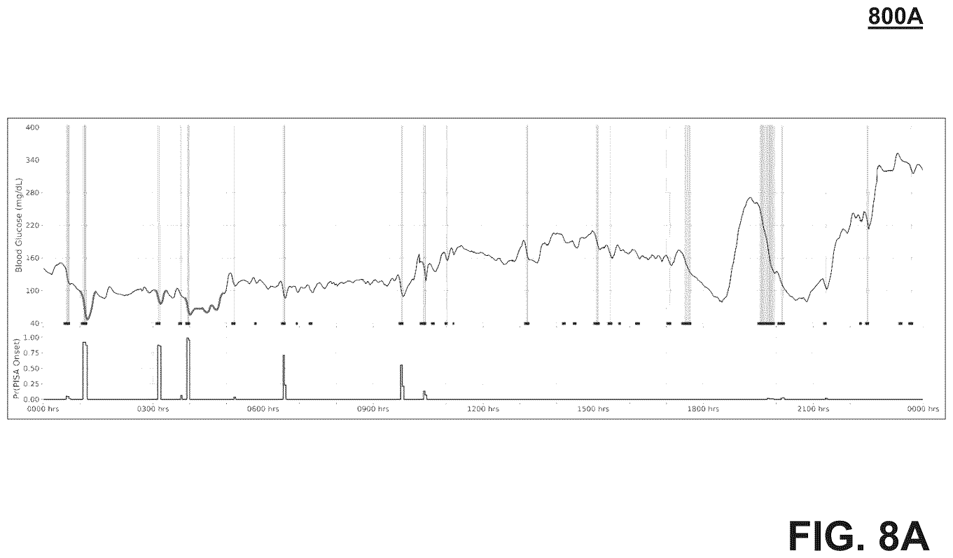

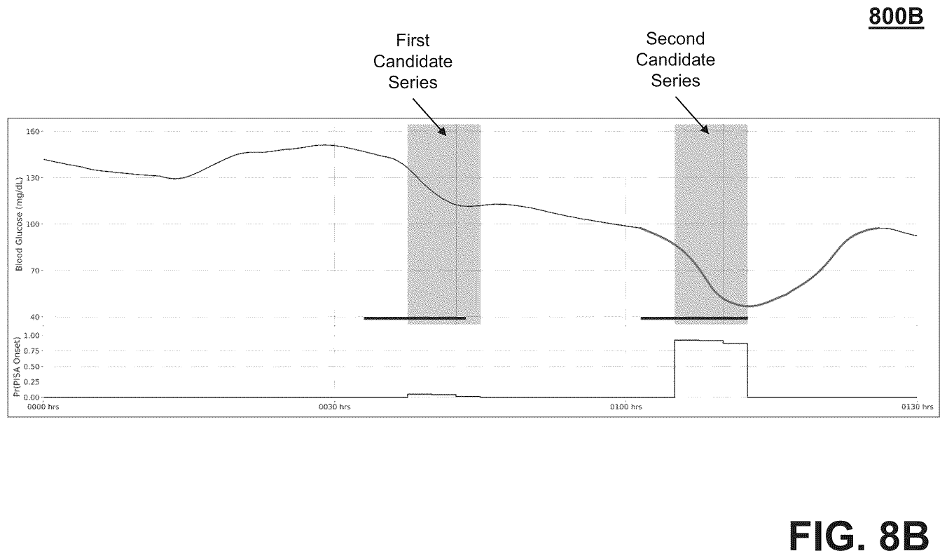

FIG. 8A shows an exemplary plot of a time series of BG measurements including plural PISAs and plural candidate series each having a drop time-window. Each candidate series in FIG. 8A is shown as having a predicted probability that the candidate series includes a PISA onset.

FIG. 8B shows an exemplary plot of a time series of BG measurements including a PISA and plural candidate series, each candidate series having a drop time-window. Each candidate series in FIG. 8B is shown having a predicted probability of PISA onset. As shown in FIG. 8B, a first candidate series is shown as being identified as having a low probability of including a PISA. A second candidate series is shown as being identified as having a high probability of including a PISA. For example, the second candidate series is determined to be a time series sub-sequence of BG measurements that was collected by a sensor that was subject to compression.

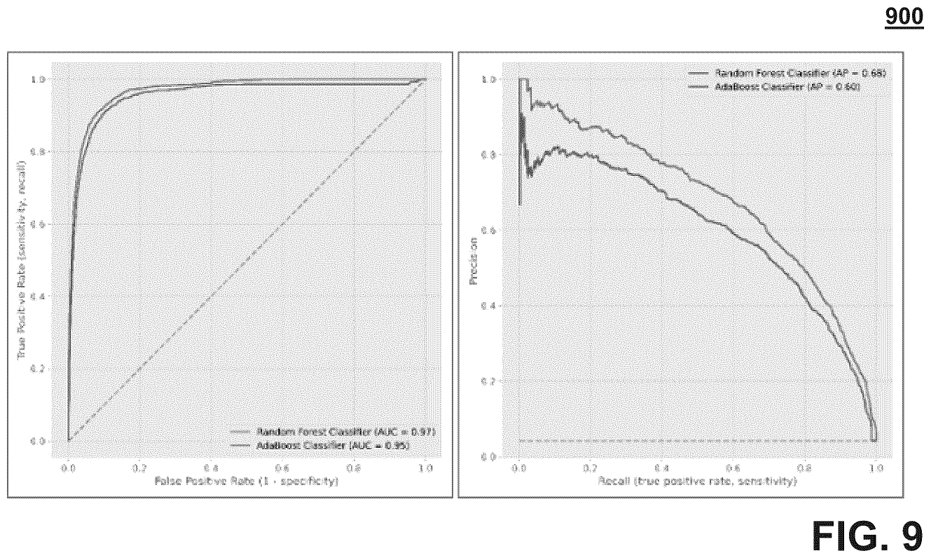

FIG. 9 shows an exemplary plot of area under the receiver operating characteristics (ROC) curve and a precision-recall curve for a classifier model that may be trained and/or used to classify time series of BG measurements as including an onset of a PISA to detect sensor compression. FIG. 9 shows how embodiments may produce at least one machine learning model that may effectively classify and/or detect PISAs in BG measurement data (e.g., in real time). A classifier model (e.g., a machine learning model) may include a random forest classifier, an adaptive boosting model (e.g., AdaBoost), and/or another machine learning algorithm trained using one or more training datasets.

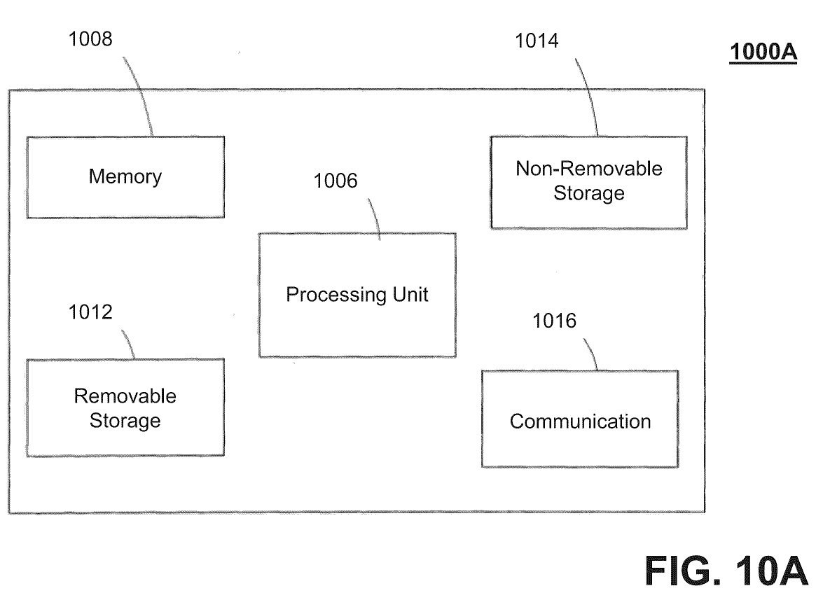

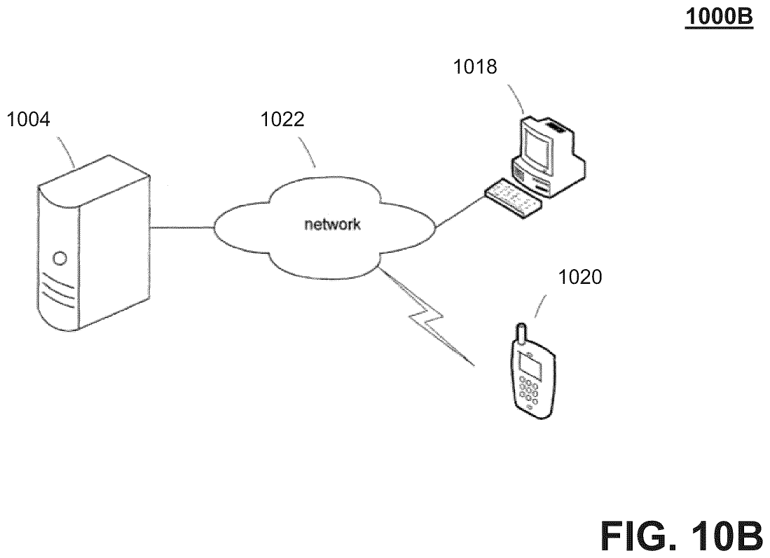

FIG. 10A shows an exemplary system configuration 1000A for an exemplary computing device (e.g., computing device 104). System configuration 1000A may include processing unit 1006, memory 1008, removable storage 1012, non-removable storage 1014, and communication interface 1016. Processing unit 1006 may be the same as or similar to processor 106 and/or processor 306. Memory 1008 may be the same as or similar to memory 108. System configuration 1000A for a computing device (e.g., computing device 104) may include at least one processing unit 1006 and memory 1008. In some embodiments, memory 1008 may include volatile (such as random access memory (RAM)), non-volatile (such as read only memory (ROM), flash memory, etc.), and/or any combination thereof.

Additionally, system configuration 1000A may include other features and/or functionality. For example, system configuration 1000A may include additional removable storage 1012 and/or non-removable storage 1014 including, but not limited to, magnetic or optical disks or tape, as well as writable electrical storage media. Computer storage media may include volatile and nonvolatile, removable and non-removable media implemented in any method or technology for storage of information such as computer readable instructions, data structures, program modules, and/or other data. Memory 1008, removable storage 1012, and non-removable storage 1014 are all examples of computer storage media. Computer storage media may include, but is not limited to, RAM, ROM, erasable programmable read only memory (EEPROM), flash memory or other memory technology compact disc read only memory (CDROM), digital versatile disks (DVD) or other optical storage, magnetic cassettes, magnetic tape, magnetic disk storage or other magnetic storage devices, or any other medium which can be used to store the desired information and which can accessed by a computing device (e.g., computing device 104). Any such computer storage media may be part of, or used in conjunction with, a computing device (e.g., computing device 104).

System configuration 1000A for an exemplary computing device may include one or more communication interfaces 1016 that allows a computing device (e.g., computing device 104) to communicate with other devices (e.g., other computing devices). Communication interface 1016 may transmit and/or carry information and/or signals in a communication media. Communication media may embody computer readable instructions, data structures, program modules and/or other data in a modulated data signal such as a carrier wave or other transport mechanism and may include any information delivery media. The term modulated data signal may refer to a signal that has one or more of its characteristics set or changed in such a manner as to encode, execute, and/or process information in the signal. By way of example, and not limitation, communication media may include wired media such as a wired network or direct-wired connection, and wireless media such as radio, RF, infrared and/or other wireless media. As discussed, the term computer readable media as used herein may include both storage media and/or communication media.

The number and arrangement of systems, hardware, devices and/or modules (e.g., software instructions) shown in FIG. 10A is provided as an example. There may be additional systems, hardware, devices, and/or modules, fewer systems, hardware, devices, and/or modules, different systems, hardware, devices, and/or modules, or differently arranged systems, hardware, devices, and/or modules than those shown in FIG. 10A. Furthermore, two or more systems, hardware, devices, and/or modules shown in FIG. 10A may be implemented within a single system, hardware, device, and/or module. A single system, hardware, device, and/or module shown in FIG. 10A may be implemented as multiple, distributed systems, hardware, devices, and/or modules. Additionally or alternatively, a set of systems, a set of hardware, a set of devices, and/or a set of modules (e.g., one or more systems, one or more hardware devices, one or more devices, one or more modules) of FIG. 10A may perform one or more functions described as being performed by another set of systems, another set of hardware, another set of devices, or another set of modules of FIG. 10A.

FIG. 10B shows an exemplary system environment 1000B in which systems, methods, devices, and/or computer-readable medium may be implemented. System environment 1000B may include at least one server 1004 (e.g., a network server), at least one client device 1018, a mobile device 1020, and a communication network 1022. In some embodiments, server 1004 may be the same as or similar to computing device 104. Client device 1018 may be the same as or similar to computing device 104.

FIG. 10B may include a network system including a plurality of computing devices (e.g., computing devices 104, servers 1004, and/or client devices 1018) that are in communication with a networking means (e.g., communication network 1022), such as a network with an infrastructure or an ad hoc network. The network connection may be wired connections and/or wireless connections to a plurality of computing devices. As an example, FIG. 10B shows a system environment including a network system in which embodiments may be implemented. In this example, a network system may include server 1004 (e.g. a network server), communication network 1022 (e.g. wired and/or wireless connections), client device 1018, and mobile device (e.g. a smart-phone) 1020 (or other handheld or portable device, such as a cell phone, laptop computer, tablet computer, GPS receiver, mp3 player, handheld video player, pocket projector, etc. or handheld devices (or non portable devices) with combinations of such features). In some embodiments, it should be appreciated that server 1004, client device 1018, and/or mobile device 1020 may include a glucose monitoring device (e.g., glucose monitoring device 302). In some embodiments, it should be appreciated that server 1004, client device 1018, and/or mobile device 1020 may include a glucose monitoring device (e.g., glucose monitoring device 302), artificial pancreas, and/or an insulin device (e.g., insulin device 304), or other interventional or diagnostic device. Any of the components shown or discussed with FIG. 10B may be multiple in number. Some embodiments may be implemented in any one of the devices of FIG. 10B. For example, execution of instructions or other desired processing may be performed on a same computing device that is any one of server 1004, client device 1018, and/or mobile device 1020. Alternatively, some embodiments may be implemented and/or performed on different computing devices of the network system shown in FIG. 10B. For example, certain desired or required processing or execution may be performed on one of the computing devices of the network (e.g., server 1004, client device 1018, mobile device 1020, and/or a glucose monitoring device), whereas other processing and execution may be performed at another computing device (e.g., server 1004, client device 1018, and/or mobile device 1020) of the network system, or vice versa. In some embodiments, certain processing and/or execution may be performed at one computing device (e.g., server 1004, client device 1018, mobile device 1020, and/or insulin device, artificial pancreas, or glucose monitor device (or other interventional or diagnostic device)); and the other processing and/or execution (e.g., of software instructions, PISA detection system 102, and/or the like) may be performed at different computing devices that may or may not be part of a network system. For example, certain processing may be performed at client device 1018, while the other processing and/or instructions are passed to server 1004 and/or mobile device 1020 where a portion of software instructions (e.g., PISA detection system 102) may be executed. This scenario may be appropriate where mobile device 1020, for example, accesses to communication network 1022 through client device 1018 (or an access point in an ad hoc network). For another example, software instructions to be protected can be executed, encoded, and/or processed with one or more embodiments. The processed, encoded and/or executed software may then be distributed to one or more customers (e.g., client devices 1018, mobile devices 1020, and/or glucose monitoring devices 302 of customers and/or subjects). Distribution of software instructions (e.g., software modules and/or software packages) may be in a form of storage media (e.g. disk) or electronic copy.

The number and arrangement of systems, hardware, devices and/or modules (e.g., software instructions) shown in FIG. 10B is provided as an example. There may be additional systems, hardware, devices, and/or modules, fewer systems, hardware, devices, and/or modules, different systems, hardware, devices, and/or modules, or differently arranged systems, hardware, devices, and/or modules than those shown in FIG. 10B. Furthermore, two or more systems, hardware, devices, and/or modules shown in FIG. 10B may be implemented within a single system, hardware, device, and/or module. A single system, hardware, device, and/or module shown in FIG. 10B may be implemented as multiple, distributed systems, hardware, devices, and/or modules. Additionally or alternatively, a set of systems, a set of hardware, a set of devices, and/or a set of modules (e.g., one or more systems, one or more hardware devices, one or more devices, one or more modules) of FIG. 10B may perform one or more functions described as being performed by another set of systems, another set of hardware, another set of devices, or another set of modules of FIG. 10B.

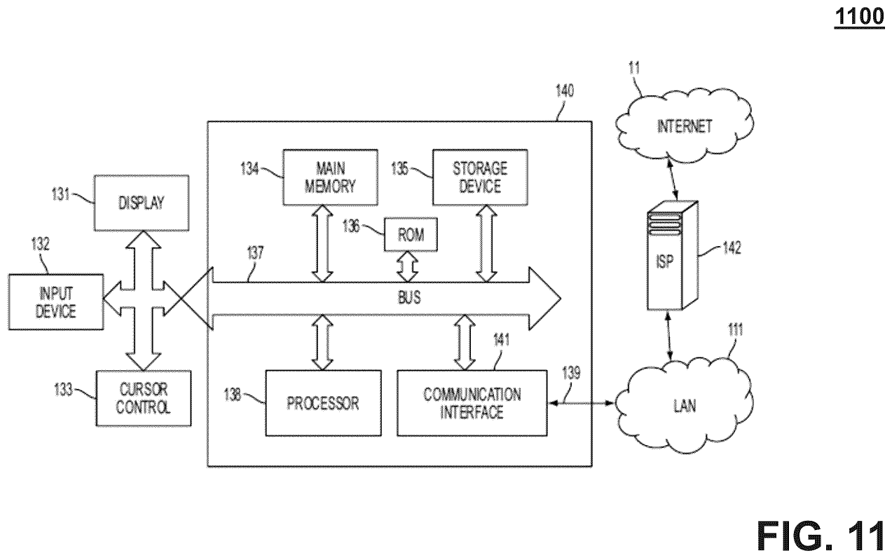

FIG. 11 is a block diagram that illustrates a system 1100 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 11. In some embodiments, computer system 140 may be the same or similar to computing device 104. The system 1100 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 11 shows various components of a computer system, it is not intended to represent any particular architecture or manner of interconnecting the components; as such details are not germane to the present disclosure. It will also be appreciated that network computers, handheld computers, cell phones and other data processing systems which have fewer components or perhaps more components may also be used. The computer system of FIG. 11 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as RAM or other dynamic storage device, coupled to bus 137 for storing information and instructions to be executed by processor 138. In some embodiments, processor 138 may be the same as or similar to processor 106 and/or processor 306.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically, computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term processor is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments are not limited to any specific combination of hardware circuitry and software.

The term computer-readable medium (or machine-readable medium) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that include bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (02-20-04), which is incorporated in its entirety for all purposes as if fully set forth herein.

Wireless links may also be implemented. In any such implementation, communication interface 141 sends and receives electrical, electromagnetic or optical signals that carry digital data streams representing various types of information.

Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

The concept of: a) detection of CGM sensor PISAs, b) improving the accuracy of CGM sensors or sensing by detecting compression artifacts inherent with devices such as medical and medicine devices and/or c) improving the action of continuous subcutaneous insulin infusion therapy and related systems, such as sensor-augmented pump (SAP), low glucose suspend (LGS), predictive low glucose suspend (PLGS), or automated insulin delivery (AID), known as an artificial pancreas are included as embodiments of the present disclosure. As provided by embodiments discussed herein, embodiments are applicable to devices for: a) providing single and/or multi-signal detection of CGM sensor PISAs, b) improving the accuracy of CGM sensors or sensing by detecting compression artifacts inherent with devices such as medical and medicine devices and/or c) improving the action of continuous subcutaneous insulin infusion therapy and related systems, such as sensor-augmented pump (SAP), low glucose suspend (LGS), predictive low glucose suspend (PLGS), or automated insulin delivery (AID), known as the “artificial pancreas”, and may be implemented and utilized with the related processors, networks, computer systems, internet, and components and functions according to embodiments disclosed herein.

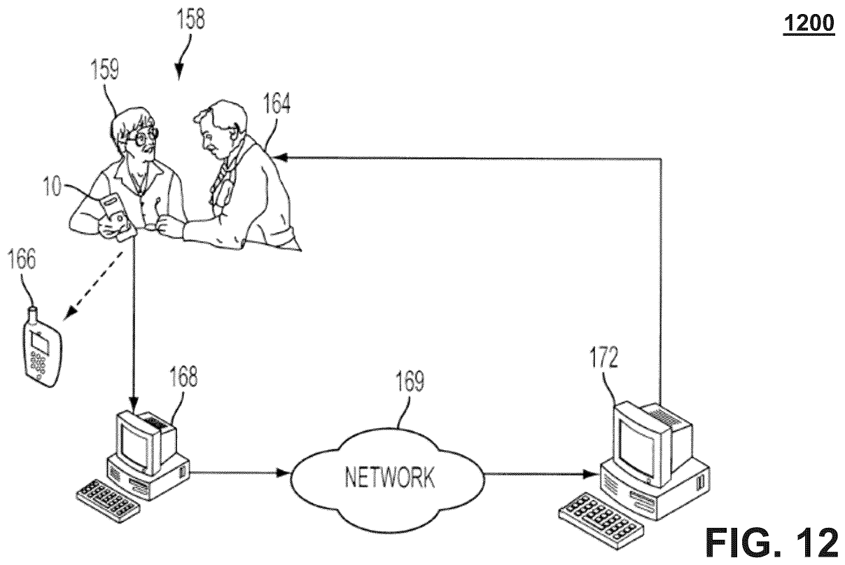

FIG. 12 shows an exemplary system and/or network in which embodiments may be implemented. In an embodiment the glucose monitor (e.g., glucose monitoring device 302), artificial pancreas or insulin device (e.g., insulin device 304) (or other interventional or diagnostic device) may be implemented by a subject (or patient) locally at home or other desired location. However, in an alternative embodiment, a glucose monitor may be implemented in a clinic setting or assistance setting. For instance, referring to FIG. 12, a clinic setup 158 provides a place for doctors (e.g. 164) or clinician/assistant to diagnose patients (e.g. 159) with diseases related with glucose and related diseases and conditions. A glucose monitoring device 10 can be used to monitor and/or test the glucose levels of the patient—as a standalone device. In some embodiments, glucose monitoring device 10 may be the same as or similar to glucose monitoring device 302. A system or component of FIG. 12 (e.g., glucose monitoring device 10) may be affixed to the patient or in communication with the patient as desired or required. For example the system or combination of components thereof—including a glucose monitor device 10 (or other related devices or systems such as a controller, and/or an artificial pancreas, an insulin pump (or other interventional or diagnostic device), or any other desired or required devices or components)—may be in contact, communication or affixed to the patient through tape or tubing (or other medical instruments or components) or may be in communication through wired or wireless connections. Such monitor and/or test can be short term (e.g., clinical visit) or long term (e.g., clinical stay or family). Glucose monitoring device outputs may be used by a doctor (clinician or assistant) for appropriate actions, such as insulin injection or food feeding for a patient, or other appropriate actions or modeling. Alternatively, the glucose monitoring device output can be delivered to computer terminal 168 for instant or future analyses. The delivery can be through cable or wireless or any other suitable medium. The glucose monitoring device output from the patient can also be delivered to a portable device, such as mobile device 166. The glucose monitoring device outputs with improved accuracy can be delivered to a glucose monitoring center 172 for processing and/or analyzing. Such delivery can be accomplished in many ways, such as network connection 170, which can be wired or wireless.

In addition to the glucose monitoring device outputs, errors, parameters for accuracy improvements, and any accuracy related information can be delivered, such as to computer 168, and/or glucose monitoring center 172 for performing error analyses. This can provide a centralized accuracy monitoring, modeling and/or accuracy enhancement for glucose centers (or other interventional or diagnostic centers), due to the importance of the glucose sensors (or other interventional or diagnostic sensors or devices).

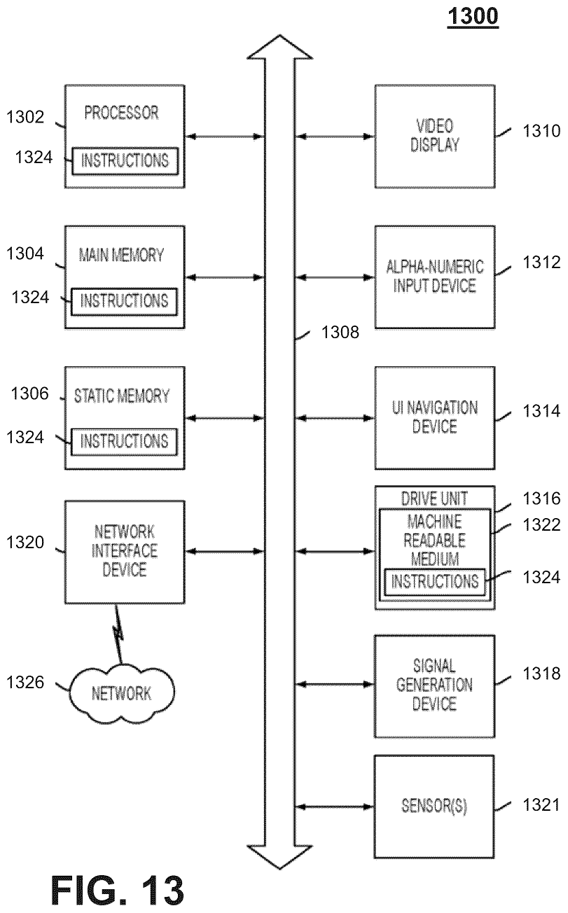

FIG. 13 is a block diagram showing an example of a machine 1300 upon which one or more aspects of embodiments may be implemented. Machine 1300 may include, but is not limited thereto, a system, method, and computer readable medium that provides for: a) multi-signal detection of CGM sensor PISAs, b) improvement of the accuracy of CGM sensors or sensing by detecting compression artifacts inherent with devices such as medical and medicine devices and/or c) improvement of the action of continuous subcutaneous insulin infusion therapy and related systems, such as sensor-augmented pump (SAP), low glucose suspend (LGS), predictive low glucose suspend (PLGS), or automated insulin delivery (AID), known as the “artificial pancreas”, which illustrates a block diagram of an example machine 1300 upon which one or more embodiments (e.g., discussed methodologies) can be implemented (e.g., run).

Examples of machine 1300 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can include dedicated circuitry or logic that is specifically configured to perform one or more techniques such as discussed above, such as including a special-purpose processor, a field programmable gate array (FPGA) or an application-specific integrated circuit (ASIC). In an example, a circuit can include programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term circuit may refer to a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits may include a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can include processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a cloud computing environment or as software as a service (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 1300) and software architectures that can be deployed in example embodiments.

In an example, the machine 1300 can operate as a standalone device or the machine 1300 can be connected (e.g., networked) to other machines.

In a networked deployment, the machine 1300 can operate in the capacity of either a server or a client machine in server-client network environments. In an example, machine 1300 can act as a peer machine in peer-to-peer (or other distributed) network environments. The machine 1300 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 1300. Further, while only a single machine 1300 is illustrated, the term machine may include any collection of machines that individually or jointly execute a set (or multiple sets) of instructions to perform any one or more of the methodologies discussed herein.

Example machine (e.g., computer system) 1300 can include a processor 1302 (e.g., a central processing unit (CPU), a graphics processing unit (GPU) or both), a main memory 1304 and a static memory 1306, some or all of which can communicate with each other via a bus 1308. The machine 1300 can further include a display unit 1310, an alphanumeric input device 1312 (e.g., a keyboard), and a user interface (UI) navigation device 411 (e.g., a mouse). In an example, the display unit 1310, input device 1312 and UI navigation device 1314 can be a touch screen display. The machine 1300 can additionally include a storage device (e.g., drive unit) 1316, a signal generation device 1318 (e.g., a speaker), a network interface device 1320, and one or more sensors 1321, such as a global positioning system (GPS) sensor, compass, accelerometer, or other sensor. In some embodiments, sensor 1321 may be the same as or similar to sensor 110.

The storage device 1316 can include a machine readable medium 1322 on which is stored one or more sets of data structures or instructions 1324 (e.g., software) embodying or utilized by any one or more of the methodologies or functions described herein. The instructions 1324 can also reside, completely or at least partially, within the main memory 1304, within static memory 1306, or within the processor 1302 during execution thereof by the machine 1300. In an example, one or any combination of the processor 1302, the main memory 1304, the static memory 1306, or the storage device 1316 can constitute machine readable media.

While the machine readable medium 1322 is illustrated as a single medium, the term machine readable medium can include a single medium or multiple media (e.g., a centralized or distributed database, and/or associated caches and servers) that configured to store the one or more instructions 1324. The term machine readable medium can also include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term machine readable medium can include, but is not limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1324 can further be transmitted or received over a communications network 1326 using a transmission medium via the network interface device 1320 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term transmission medium may include any intangible medium that is capable of storing, encoding or carrying instructions for execution by the machine, and includes digital or analog communications signals or other intangible medium to facilitate communication of such software.

Any of the processors disclosed herein can be part of or in communication with a machine (e.g., a computer device, a logic device, a circuit, an operating module (hardware, software, and/or firmware), etc.). The processor can be hardware (e.g., processor, integrated circuit, central processing unit, microprocessor, core processor, computer device, etc.), firmware, software, etc. configured to perform operations by execution of instructions embodied in computer program code, algorithms, program logic, control, logic, data processing program logic, artificial intelligence programming, machine learning programming, artificial neural network programming, automated reasoning programming, etc. The processor can receive, process, and/or store data.

Any of the processors disclosed herein can be a scalable processor, a parallelizable processor, a multi-thread processing processor, etc. The processor can be a computer in which the processing power is selected as a function of anticipated network traffic (e.g., data flow). The processor can include an integrated circuit or other electronic device (or collection of devices) capable of performing an operation on at least one instruction, which can include a Reduced Instruction Set Core (RISC) processor, a Complex Instruction Set Computer (CISC) microprocessor, a Microcontroller Unit (MCU), a CISC-based Central Processing Unit (CPU), a Digital Signal Processor (DSP), a Graphics Processing Unit (GPU), a Field Programmable Gate Array (FPGA), etc. The hardware of such devices may be integrated onto a single substrate (e.g., silicon “die”), distributed among two or more substrates, etc. Various functional aspects of the processor may be implemented solely as software or firmware associated with the processor.

The processor can include one or more processing or operating modules. A processing or operating module can be a software or firmware operating module configured to implement any of the functions disclosed herein. The processing or operating module can be embodied as software and stored in memory, the memory being operatively associated with the processor. A processing module can be embodied as a web application, a desktop application, a console application, etc.

The processor can include or be associated with a computer or machine readable medium. The computer or machine-readable medium can include memory. Any of the memory discussed herein can be computer readable memory configured to store data. The memory can include a volatile or non-volatile, transitory or non-transitory memory, and be embodied as an in-memory, an active memory, a cloud memory, etc. Examples of memory can include flash memory, Random Access Memory (RAM), Read Only Memory (ROM), Programmable Read only Memory (PROM), Erasable Programmable Read only Memory (EPROM), Electronically Erasable Programmable Read only Memory (EEPROM), FLASH-EPROM, Compact Disc (CD)-ROM, Digital Optical Disc DVD), optical storage, optical medium, a carrier wave, magnetic cassettes, magnetic tape, magnetic disk storage or other magnetic storage devices, or any other medium which can be used to store the desired information and which can accessed by the processor.

The memory can be a non-transitory computer-readable medium. The term computer-readable medium (or machine-readable medium) as used herein is an extensible term that refers to any medium or any memory, that participates in providing instructions to the processor for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, transmission media, etc. The computer or machine readable medium can be configured to store one or more instructions or computer programs thereon. The instructions or computer programs can be in the form of algorithms, program logic, etc. that cause the processor to execute any of the functions disclosed herein.

Embodiments of the memory can include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc. Communications can be via Bluetooth, near field communications, cellular communications, telemetry communications, Internet communications, etc.

Transmission of data and signals can be via transmission media. Transmission media can include coaxial cables, copper wire, fiber optics, etc. Transmission media can also take the form of acoustic or light waves, such as those generated during radio-wave and infrared data communications, or other form of propagated signals (e.g., carrier waves, digital signals, etc.).

Any of the processors can be in communication with other processors of other devices (e.g., a computer device, a computer system, a laptop computer, a desktop computer, etc.). For instance, the processor of the system configuration 100 can be in communication with a processor of another computing device 104, the processor of the computing device 104 can be in communication with a processor of a display, sensor 110, etc. Any of the processors can have transceivers or other communication devices/circuitry to facilitate transmission and reception of wireless signals. Any of the processors can include an Application Programming Interface (API) as a software intermediary that allows two or more applications to talk to each other. Use of an API can allow software of one processor to communicate with software of another processor of another device(s).

Any of the data or communication transmissions between two components can be a push operation and/or a pull operation. For instance, data transfer between the processor 106 and the memory 108 or the processor 106 and sensor 110, etc. can be push operation (e.g., the data can be pushed from the memory) and/or a pull operation (e.g., the processor can pull the data from the memory), data transfer between another system and computing device 104 can be a push and/or pull operation, etc.

When data is received by a component, it can be processed in real time, stored in memory for later processing, or some combination of both. After being processed by the component, the processed data can be used in real time, stored in memory for later use, or some combination of both. The pre-processed data and/or the processed data can be encoded, tagged, or labeled before, during, or after being stored in memory.

As noted herein, the system configuration 100 can include memory 108 containing a computer program (e.g., software instructions for PISA detection system 102) that when executed can cause the processor 106 to perform any of the functions/operations disclosed herein.

The computer program can cause the processor to execute one or more machine learning models (e.g., linear regression model, tree based model, perceptron based model, Gaussian based model, etc.). It is contemplated for at least one of the machine learning models to be a random forest or AdaBoost machine learning model.

### EXAMPLES

The following describes exemplary methods and systems developed, along with test results for the exemplary methods and systems.

An aspect of an embodiment the presently disclosed method, system and computer readable medium provides, among other things, the ability to detect CGM sensor compression lows in real time and thereby prevent compression low effects impacting negatively the treatment of diabetes, such as false hypoglycemia alarms, or insulin shutoff by insulin delivery systems. For the purpose of an aspect of an embodiment, an insulin delivery system can be: (i) Sensor-augmented pump (SAP) therapy; (ii) Low glucose suspend (LGS) system or Predictive low glucose suspend system (PLGS), or (iii) Automated insulin delivery (AID), known as an “artificial pancreas.”

An aspect of an embodiment the presently disclosed method, system and computer readable medium includes, among other things, two algorithms which work in tandem to fully identify PISAs in a prospective manner. The first algorithm is a prospective single-sensor algorithm used to detect the onset of a PISA. The second algorithm is a retrospective single-sensor algorithm used to detect the end of a PISA. Both algorithms make use of machine learning techniques to learn from the input data and in this embodiment, they use Random Forests and AdaBoost models. In other alternative embodiments the machine learning algorithms could be, but are not limited to, Gradient Boosted Trees, Neural Networks, Support Vector Machines, etc., or any combination thereof.

In an embodiment, internally, the method, system and computer readable medium use one or more signals available with CGM sensors, which include but are not limited to: raw blood glucose estimate (prior to calibration and temperature corrections), filtered blood glucose, temperature, time of day, time of sensor life, and/or any combination thereof. These signals, when available, are supplied to machine learning techniques and/or models. Externally, the method can utilize additional information from other PISA detection techniques, considerations regarding glycemic events such as hypoglycemia, or data from anticipated future sensors, such as a pressure sensor added to the CGM sensor. In an embodiment, the external update of the method, system and computer readable medium with additional information/signals is done via an iterative Bayesian update procedure.

In an embodiment, to develop the method, system and computer readable medium, the algorithms in one embodiment were trained on CGM data traces from 67 individuals containing 58,403 hours of sensor data and 1,089 PISA events and were then tested on an independent testing data set with data from 44 individuals, 36,206 hours of CGM data, and 534 PISA events.

### Data Overview

An exemplary data set included 407 files generated by 113 subjects. An embodiment may include two subjects with just one sensor, with 111 subjects with usable data: concurrent CGM data from 2 or more sensors. Embodiments may include: 78 subjects with 4 sensors, 27 subjects with 3 sensors, and 6 subjects with 2 sensors. The data may include <1% of sensors in warmup, 93.6% where glucose would be reported to the user, <1% of sensors experiencing some transient issue in the sensor signal, and 5.5% where the sensor experienced a failure.

Each subject may have two different sets of data. The data which resides in the ‘Display’ table may be sampled every 5 minutes, while the data which resides in the ‘Intermediate’ table may be sampled every 30 seconds. The data in the ‘Display’ table may include a subset of the data in the ‘Intermediate’ table, and therefore in this work data from the ‘Intermediate’ table may be used.

The data from the ‘Intermediate’ table may be collated so that all sensor data from all subjects is placed in a single CSV file of 12,039,945 rows and 19 columns. For a given subject, the start time of the 2 or more sensors is different so that BG values are recorded at different time stamps. In order to compare BG values across multiple sensors, the data may be resampled using the Python pandas resample function with 30-second time buckets which start at 00:00 midnight each day. Under this resampling scheme the time stamp of any BG value recorded during the 30-second time bucket is set to the start of the time bucket. For example, a BG value with timestamp 11:37:23 will have a timestamp of 11:37:00 after the re-sampling. In this way, BG values can be directly compared across sensors at any given time stamp.

For the purposes of model building the data set may be divided into a Training data set and a Testing data set. The data may be split by subject so that all sensor data from a single subject is placed into either the Training data set or the Testing data set. There may include 67 subjects (60.4%) in a Training data set and 44 subjects (39.6%) in a Testing data set in one embodiment.

The Training data set may be used to train and cross-validate the models (to set model hyperparameters, etc.) while the Testing data set may be used to test the models once the models are finalized and fixed.

An algorithm may be used to generate visualizations such that each visualization contains enough information for an individual to (subjectively) determine if the highlighted time series subsequence includes a PISA. The algorithm may inspect the BG time series and may identify a potential PISA start and all subsequent potential PISA ends. In some instances, the potential PISA start and the last potential PISA end may define a time series subsequence. Using this time series subsequence information, the algorithm may generate a visualization which highlights the sensor trace under consideration and indicates the potential PISA start and all potential PISA ends. In some embodiments, the time series subsequence which begins at the start of a drop time-window and ends at the rise end of the first rise time-window may be a PISA.

Manual review of 8,002 visualizations, generated using the BG time series from all sensors of the 111 subjects, may be used to annotate each time series with PISAs whose minimum BG value was less than 85 mg/dL (e.g., hypo- or near hypo-PISAs). This review resulted in 1,623 hypo- or near hypo-PISAs from 94,609.5 hours of sensor data. Of the 1,623 annotations, there may be about 1,089 in the Training data set (67 subjects) from 59,122 hours of sensor data. The analysis may concern just those 1,089 PISA annotations in the Training data set.

If daytime is defined as the 16-hour time period from 7:00am until 11:00pm and nighttime is defined as the 8-hour time period from 11:00pm until 7:00am, then 613 of the 1,089 PISAs occurred during the daytime while 476 began during the nighttime.

Further, 591 of the 1,089 PISAs occurred on sensors worn on the abdomen while 498 occurred on sensors worn on the arm. Of the 591 PISAs which occurred on sensors worn on the abdomen, 381 began during the daytime while 210 began during the nighttime; and of the 498 PISAs which occurred on sensors worn on the arm, 232 began during the daytime while 266 began during the nighttime.

A distribution of PISAs appears to follow an exponential distribution with the vast majority of PISAs being less than 60 minutes in length. The top three rows of Table 1 provide descriptive statistics for these distributions. There does appear to be a slight difference in distribution of the length of the PISA based on when the PISA started.

TABLE 1

| | | Mean | | | 25^{th} | | 75^{th} | |
|---|---|---|---|---|---|---|---|---|
| | | (Stdev) | | Min. | Pctl. | Median | Pctl. | Max. |
| Duration | All | 38 | (24) | 15 | 22 | 31 | 47 | 176 |
| (mins) | Daytime | 36 | (22) | 15 | 21 | 30 | 44 | 176 |
| | Nighttime | 40 | (26) | 15 | 23 | 32 | 49 | 176 |
| Starting BG | All | 100 | (31) | 40 | 83 | 98 | 112 | 400 |
| (mg/dL) | Daytime | 102 | (32) | 40 | 85 | 100 | 113 | 400 |
| | Nighttime | 97 | (28) | 40 | 81 | 97 | 112 | 344 |
| BG Delta | All | 1 | (29) | −157 | −11 | 1 | 14 | 219 |
| (mg/dL) | Daytime | 2 | (32) | −157 | −10 | 3 | 16 | 219 |
| | Nighttime | −1 | (24) | −97 | −12 | −1 | 10 | 181 |

There data appears to show that there are times of day when a PISA is more likely to start (e.g., during the time period 9:30pm through 6:00am). This is even more pronounced when considering where the sensor is worn (e.g., arm or abdomen). For example, PISAs may be more likely to occur during the initial 12 hours of sensor life. Many of these observations may be used to generate features (e.g., time of day, starting BG value, starting temperature, etc.) as input for the machine learning models using the distributions presented.

In one embodiment, the algorithm to identify PISA onset outputs a probability that a 2.5-minute time interval corresponds to the onset of a PISA. In this embodiment, the output of this algorithm is a time series of probability values (one every 2.5 minutes), where the probability is the probability that the 2.5-minute time interval is part of the onset of a PISA. In other embodiments the time interval can be different lengths (e.g., 30 seconds, 1 minute, 5 minutes) depending on the sensor sampling resolution.

In one embodiment the algorithm takes the CGM time series and, every 2.5 minutes, constructs a time series subsequence with starting time stamp equal to 31 minutes prior to the most current time stamp and ending time stamp equal to the current time stamp (e.g., the most current blood glucose value available). In other embodiments the starting time stamp can vary. The algorithm then computes a rolling mean with lag of about 1.5 minutes on the original time series subsequence, resulting in a 30-minute time series subsequence. The lag depends on the sensor sampling resolution, and can be different in other embodiments. In one embodiment this resulting time series subsequence is then used to generate a 25-minute time series subsequence of indicator (e.g., True/False) values indicating whether the difference in BG value between each time stamp and the time stamp 5 minutes ago exceeds a predetermined threshold.

If the set of time stamps which are True is such that (1) the indicator value at the most recent time stamp is True, and the set of contiguous indicator values (including the indicator value at the most recent time stamp) which are True span at least 2.5 minutes, OR (2) the indicator value at the most recent time stamp is True and the change in BG value is greater than 7.5 mg/dL (in this embodiment) then this time series subsequence is a candidate for evaluation. In other embodiments different prespecified thresholds can be used. If the time series subsequence is not a candidate for evaluation, the 2.5-minute time interval is assigned a probability of 0 (e.g., probability 0 for onset of PISA). If the time series subsequence is a candidate for evaluation, the 2.5-minute time interval is assigned the probability value from the classifier used to classify the time series subsequence.

If the time series subsequence is a candidate for evaluation (e.g., there is reason to believe it may represent the start of a PISA), a classifier is used to provide the probability of PISA onset for, in one embodiment, the 2.5-minute time interval. For a given time series subsequence, in one embodiment, groups of features are generated using all of the time series signals recorded by the CGM sensor (blood glucose, raw [e.g., non-temperature corrected] blood glucose, temperature) and used as input to the machine learning model. The features which may be generated for each time series subsequence that is a candidate for further evaluation are outlined. In other embodiments, any subset of the features described can be generated and used as input to a model.

### Features

Start Time

SL_Start_Time_Idx: Index indicating the sensor life interval into which the PISA start time falls, where in one embodiment the intervals are {0: (2, 7.5), 1: (7.5, 11), 2: (0, 2)}.

SL_Start_Time_Prob: Probability of a PISA start taken from the distribution of the sensor life start time.

ToD_Start_Time_Idx: Index indicating the time of day interval into which the PISA start time falls, where in one embodiment the intervals are {0: (19, 24), 1: (6, 19), and 2: (0, 6)}.

ToD_Start_Time_Prob: Probability of a PISA start taken from the distribution of the time of day start time.

### Blood Glucose

BG_Start: The BG value at the drop start.

BG_End: The BG value at the time series subsequence end.

BG_Drop_Delta: The difference in BG values at drop start and time series subsequence end.

BG_Drop_LRSlope: The slope of the linear regression (no fixed intercept) fit to the BG values recorded between the drop start and time series subsequence end.

BG_Drop_SD: The standard deviation of the BG values recorded between the drop start and time series subsequence end.

BG_Prev5min_SD: The standard deviation of the BG values recorded in the 5 minutes prior to the drop start.

BG_Prev10min_SD: The standard deviation of the BG values recorded in the 10 minutes prior to the drop start.

### Raw Blood Glucose

BGRaw_Drop_Delta: The difference in raw BG values at drop start and time series subsequence end.

BGRaw_Drop_LRSlope: The slope of the linear regression (no fixed intercept) fit to the raw BG values recorded between the drop start and time series subsequence end.

BGRaw_Drop_SD: The standard deviation of the raw BG values recorded between the drop start and time series subsequence end.

BGRaw_Prev5min_SD: The standard deviation of the raw BG values recorded in the 5 minutes prior to the drop start.

BGRaw_Prev10min_SD: The standard deviation of the raw BG values recorded in the 10 minutes prior to the drop start.

### Temperature

Temp_Drop_Delta: The difference in temperature values at drop start and time series subsequence end.

Temp_Prev5min_Delta: The difference in temperature values at 5 minutes prior to drop start and drop start.

Temp_Prev10min_Delta: The difference in temperature values at 10 minutes prior to drop start and drop start.

Temp_Drop_LRSlope: The slope of the linear regression (no fixed intercept) fit to the temperature values recorded between the drop start and time series subsequence end.

Temp_Drop_SD: The standard deviation of the temperature values recorded between the drop start and time series subsequence end.

Temp_Prev5min_SD: The standard deviation of the temperature values recorded in the 5 minutes prior to drop start.

Temp_Prev10min_SD: The standard deviation of the temperature values recorded in the 10 minutes prior to drop start.

Temp_Prev5min_DropEnd_Delta: The difference in temperature values at 10 minutes prior to drop start and time series subsequence end.

### Blood Glucose and Raw Blood Glucose Comparison

BGComp_Drop: The value of the norm_bg_bgraw_delta function (see below) as computed using the BG values recorded between drop start and time series subsequence end.

BGComp_Prev5min: The value of the norm_bg_bgraw_delta function (see below) as computed using the BG values recorded in the 5 minutes prior to drop start.

BGComp_Prev10min: The value of the norm_bg_bgraw_delta function (see below) as computed using the BG values recorded in the 10 minutes prior to drop start.

In one embodiment the sensor data from the 67 subjects in the Training data set may be used to generate a Training input features data set with 53,025 rows of data. This Training input features data set was used to train Random Forest and AdaBoost learning models using the scikit-learn Python package. In alternate embodiments, other machine learning algorithms including but not limited to linear regression, neural networks, or support vector machines can be trained using this Training input features data set (or Training input features data set consisting of subsets of the features described).

### Results

Embodiments may include two different performance evaluations. A first performance evaluation is the evaluation of the classifier model which classifies whether or not the 2.5-minute time interval corresponds to the onset of a PISA.

As such, in one embodiment: a positive event is a 2.5-minute time interval which overlaps in any way with the drop time-window of a PISA, and a negative event is a 2.5-minute time interval which does not overlap in any way with the drop time-window of a PISA.

In other embodiments, the time interval can have different lengths. In one embodiment the machine learning classifier models may be trained on the Training input features data set and grid search along with 5-fold cross-validation to identify the optimal hyper-parameter settings for each model (Table 2 lists the hyperparameters and, in one embodiment, possible values).

TABLE 2

| Model | Hyperparameter | Possible Values |
|---|---|---|
| Random Forest | n_estimators | 75, 100, 150 |
| | max_depth | None, 15 |
| | max_features | None, log2, sqrt |
| | class_weight | balanced, {0:1, 1:5}, {0:1, 1:15} |
| AdaBoost | n_estimators | 100, 250, 350, 450 |
| | learning_rate | 0.1, 0.5, 1 |

In one embodiment, the scoring criterion used is the F1 score, which may address both precision and recall performance of binary classifier models. In one embodiment, a Random Forest model identified had n_estimators=150, max_depth=15, max features=sqrt, and class_weight={0: 1, 1:5}, while an AdaBoost model identified had n estimators=450 and learning_rate=0.5. In other embodiments, the Training input feature data set may be used in conjunction with cross-validation to train a model and identify improved hyper-parameter settings, and area under the ROC curves and precision-recall curves can be used to assess the model performance using the Testing input features data set.

A second performance evaluation may include an evaluation of the entire single-sensor prospective algorithm which classifies whether a drop time-window was correctly classified as the onset of a PISA. As such, in one embodiment: a positive event is a drop time-window which is the drop time-window of a PISA, a negative event is a drop time-window which is not the drop time-window of a PISA.

This second evaluation considers the entire process and includes evaluating how well the initial screening process performed (where, in one embodiment, each 2.5-minute time interval was either assigned a probability of 0 for PISA onset or was sent to the classifier to determine the probability of PISA onset). During the course of the BG time series, each drop time-window is a potential PISA onset. As such, the set of drop time-windows is the set of candidate series where a classification must be made (e.g., the drop time-window is the start of PISA or not). There may be drop time-windows which do not have 2.5-minute time intervals where the time series subsequence needs further evaluation, and all 2.5-minute time intervals may overlap some portion (e.g., at least one time stamp) of a drop time-window.

For each drop time-window, in one embodiment the set of 2.5-minute time intervals which overlap in any way with the drop time-window may be used to determine the probability of the drop time-window corresponding to PISA onset by taking the maximum probability of PISA onset over all the intervals.

An algorithm to identify PISA end works retrospectively to identify if the end of a PISA has been reached. The algorithm looks at the CGM sensor time series of BG measurements and attempts to identify the most recent potential PISA start and a subsequent potential PISA end. Once a time series subsequence has been identified, the algorithm generates a number of features which can then be used as input to a machine learning model which will assign a probability to the time series subsequence, where the probability is the probability of that particular time series being a PISA. If the probability is above a certain threshold τ_PISA then the time series subsequence is classified as a PISA, and the end of the PISA has been identified.

To identify potential starts and ends of PISAs, rough estimates of two different time-windows may be obtained: (1) Drop time-window: a set of consecutive time stamps t such that (BG_t-BG_(t-lag))<τ_D where in one embodiment τ_D=10 mg/dL is the drop threshold; and (2) Rise time-window: a set of consecutive time stamps t such that (BG_t-BG_(t-lag))>τ_R where in one embodiment τ_R=6 mg/dL is the rise threshold. The variable lag represents the number of 30-second time intervals. For example, lag=10 corresponds to looking back 5 minutes. Note that both time-windows have a start (drop start and rise start) and an end (drop end and rise end).

The starts and ends of the drop and rise time-windows may need refining. In one embodiment, in each case this refinement is accomplished by first computing a rolling mean (moving average) of BG values using the most recent 90 seconds of data, and then computing the first difference of the rolling mean. In other embodiments different amounts of recent data can be used to compute the rolling mean.

The refined drop start may be defined as a first timestamp where the first difference is less than the drop threshold, δ_D, or if no such timestamp exists, then the first timestamp with a negative first difference. Otherwise, a default refined drop start may be the first timestamp of the drop time-window.

The refined drop end may be defined as a last possible timestamp where the first difference is greater than δ_D. Otherwise, a default refined drop end may be the last timestamp of the drop time-window.

Similarly, the refined rise start may be defined as a first timestamp where the first difference is greater than the rise threshold, δ_R, or if no such timestamp exists, then the first timestamp with a positive first difference. Otherwise, a default refined rise start may be the first timestamp of the rise time-window.

The refined rise end may be defined as the last possible timestamp where the first difference is less than δ_R. Otherwise a default refined rise end may be the last timestamp of the rise time-window.

One of the defining characteristics of a PISA may include a “sudden” drop of at least 20 mg/dL. Thus, in one embodiment any drop time-window in which the change in BG value between the drop start and the drop end is at least 20 mg/dL is the potential start of a PISA.

The set of rise time-windows that may be potential matches for a given drop time-window are those rise time-windows where, in one embodiment, the rise end is at least 15 minutes after and no more than 180 minutes after the drop start.

Given a drop time-window and a potential matching rise time-window (e.g., a PISA candidate), it is possible to calculate the minimum BG value between the drop start and the rise end. In one embodiment, only PISA candidates where the minimum BG value is less than 85 mg/dL are kept.

For a given time series subsequence, in one embodiment, groups of features may be generated using all of the time series signals recorded by the CGM sensor (blood glucose, raw [e.g., non-temperature corrected] blood glucose, temperature) and used as input to the machine learning model. The following subsections outline the features which are generated for each time series subsequence that is a candidate for further evaluation. In other embodiments, any subset of the features described may be generated and used as input to a model.

Rise_Match_Idx: The index of the rise time-window (based on all of the rise time-windows which matched the drop time-window).

### Start Time

SL_Start_Time_Idx: Index indicating the sensor life interval into which the PISA start time falls, where in one embodiment the intervals are {0: (2, 7.5), 1: (7.5, 11), 2: (0, 2)}.

SL_Start_Time_Prob: Probability of a PISA start taken from the distribution of the sensor life start time.

ToD_Start_Time_Idx: Index indicating the time of day interval into which the PISA start time falls, where in one embodiment the intervals are {0: (19, 24), 1: (6, 19), and 2: (0, 6)}.

ToD_Start_Time_Prob: Probability of a PISA start taken from the distribution of the time of day start time.

### Duration

Duration_Drop_mins: The duration of the drop time-window in minutes.

Duration_Drop_Prob: Probability of a drop time-window of this duration by time of day taken from the distribution of drop time-window duration.

Duration_Rise_mins: The duration of the rise time-window in minutes.

Duration_Rise_Prob: Probability of a rise time-window of this duration by time of day taken from the distribution of rise time-window duration.

Duration_PISA_mins: The duration of the PISA in minutes.

Duration_PISA_Prob: Probability of a PISA of this duration by time of day taken from the distribution of PISA duration.

### Blood Glucose

BG_Start: The BG value at drop start.

BG_Start_Prob: Probability of this starting BG value by time of day.

BG_Minimum: The minimum BG value between drop start and rise end.

BG_Drop_Delta: The difference in BG values at drop start and drop end.

BG_Rise_Delta: The difference in BG values at rise start and rise end.

BG_PISA_Delta: The difference in BG values at drop start and rise end.

BG_Drop_LRSlope: The slope of the linear regression (no fixed intercept) fit to the BG values recorded between the drop start and drop end.

BG_Rise_LRSlope: The slope of the linear regression (no fixed intercept) fit to the BG values recorded between the rise start and rise end.

BG_Drop_SD: The standard deviation of the BG values recorded between the drop start and drop end.

BG_Rise_SD: The standard deviation of the BG values recorded between the rise start and rise end.

BG_PISA_SD: The standard deviation of the BG values recorded between the drop start and rise end.

BG_Prev5min_SD: The standard deviation of the BG values recorded in the 5 minutes prior to the drop start.

BG_Prev10min_SD: The standard deviation of the BG values recorded in the 10 minutes prior to the drop start.

BG_ExpRatio_2min: The value of the expected ratio BG function (see below) as computed using the 2 minutes of BG values recorded prior to drop start.

BG_ExpRatio_5min: The value of the expected ratio BG function (see below) as computed using the 5 minutes of BG values recorded prior to drop start.

BG_ExpRatio_10min: The value of the expected ratio BG function (see below) as computed using the 10 minutes of BG recorded values prior to drop start.

### Raw Blood Glucose

BGRaw_Drop_Delta: The difference in raw BG values at drop start and drop end.

BGRaw_Rise_Delta: The difference in raw BG values at rise start and rise end.

BGRaw_PISA_Delta: The difference in raw BG values at drop start and rise end.

BGRaw_Drop_LRSlope: The slope of the linear regression (no fixed intercept) fit to the raw BG values recorded between the drop start and drop end.

BGRaw_Rise_LRSlope: The slope of the linear regression (no fixed intercept) fit to the raw BG values recorded between the rise start and rise end.

BGRaw_Drop_SD: The standard deviation of the raw BG values recorded between the drop start and drop end.

BGRaw_Rise_SD: The standard deviation of the raw BG values recorded between the rise start and rise end.

BG_PISA_SD: The standard deviation of the raw BG values recorded between the drop start and rise end.

BGRaw_Prev5min_SD: The standard deviation of the raw BG values recorded in the 5 minutes prior to the drop start.

BGRaw_Prev10min_ SD: The standard deviation of the raw BG values recorded in the 10 minutes prior to the drop start.

### Temperature

Temp_Drop_Delta: The difference in temperature values at drop start and drop end.

Temp_Rise_Delta: The difference in temperature values at rise start and rise end.

Temp_PISA_Delta: The difference in temperature values at drop start and rise end.

Temp_Prev5min_Delta: The difference in temperature values at 5 minutes prior to drop start and drop start.

Temp_Prev10min_Delta: The difference in temperature values at 10 minutes prior to drop start and drop start.

Temp_Drop_LRSlope: The slope of the linear regression (no fixed intercept) fit to the temperature values recorded between the drop start and drop end.

Temp_Rise_LRSlope: The slope of the linear regression (no fixed intercept) fit to the temperature values recorded between the rise start and rise end.

Temp_PISA_LRSlope: The slope of the linear regression (no fixed intercept) fit to the temperature values recorded between the drop start and rise end.

Temp_Drop_SD: The standard deviation of the temperature values recorded between the drop start and drop end.

Temp_Rise_SD: The standard deviation of the temperature values recorded between the rise start and rise end.

Temp_PISA_SD: The standard deviation of the temperature values recorded between the drop start and rise end.

Temp_Prev5min_SD: The standard deviation of the temperature values recorded in the 5 minutes prior to drop start.

Temp_Prev10min_SD: The standard deviation of the temperature values recorded in the 10 minutes prior to drop start.

Temp_ DropEnd_MinBGIdx_Delta: The difference in temperature values at drop end and the time stamp of the minimum BG value during the PISA.

Temp_Prev5min_DropEnd_Delta: The difference in temperature values at 5 minutes prior to drop start and drop end.

Temp_Prev5min_RiseStart_Delta: The difference in temperature values at 5 minutes prior to drop start and rise start.

Temp_DropEnd_RiseEnd_Delta: The difference in temperature values at drop end and rise end.

### Blood Glucose and Raw Blood Glucose Comparison

BGComp_Drop: The value of the norm_bg_bgraw_delta function (see below) as computed using the BG values recorded between drop start and drop end.

BGComp_Rise: The value of the norm_bg_bgraw_delta function (see below) as computed using the BG values recorded between rise start and rise end.

BGComp_PISA: The value of the norm_bg_bgraw_delta function (see below) as computed using the BG values recorded between drop start and rise end.

BGComp_Prev5min: The value of the norm_bg_bgraw_delta function (see below) as computed using the BG values recorded in the 5 minutes prior to drop start.

BGComp_Prev10min: The value of the norm_bg_bgraw_delta function (see below) as computed using the BG values recorded in the 10 minutes prior to drop start.

In one embodiment, the sensor data from the 67 subjects in the Training data set may be used to generate a Training input features data set with many rows of data (e.g., 18,948 rows of data). This Training input features data set may be used to train Random Forest and AdaBoost learning models using the scikit-learn Python package. In alternate embodiments other machine learning algorithms including but not limited to linear regression, neural networks, or support vector machines can be trained using this Training input features data set (or Training input features data set including subsets of the features described).

A performance evaluation of the retrospective single-sensor algorithm evaluates the classifier model which classifies whether or not a time series subsequence defined by a drop time-window and a matching rise time-window corresponds to a PISA. As such, in one embodiment: a positive event is a time series subsequence defined by a drop time-window and matching rise time-window which is a PISA, and a negative event is a time series subsequence defined by a drop time-window and matching rise time-window which is not a PISA.

In one embodiment the machine learning classifier models may be trained on the Training input features data set and grid search along with 5-fold cross-validation to identify the optimal hyper-parameter settings for each model (Table 2 lists the hyperparameters and, in one embodiment, possible values). In one embodiment, the scoring criterion may include the F1 score, which addressing both precision and recall performance of binary classifier models. In one embodiment, a Random Forest model identified had_n estimators=150, max_depth=15, max_features=sqrt, and class_weight={0: 1, 1:15}, while an AdaBoost model identified had n_estimators=100 and learning rate=1. In other embodiments the Training input features data set can be used in conjunction with cross-validation to train a model and identify improved hyper-parameter settings, and area under the ROC curves and precision-recall curves can be used to assess the model performance on the Testing input features data set.

Embodiments may use signals typically available from CGM sensors, such as raw blood glucose estimates, filtered blood glucose, temperature, as well as corrections for time of day and/or sensor life. Because more than one machine learning technique may be used for retrieval of these data and because other signals/considerations may influence the decision to detect and/or not flag a PISA event (e.g., probability for impending hypoglycemia), embodiments may provide the following scheme for utilization of external data and considerations.

A Bayesian combination of the probabilistic output from multiple models can be used when this information is available or relevant. In one embodiment, the Bayesian combination is constructed from the time series of probabilities from the Random Forest model and the time series of probabilities from the AdaBoost model. In alternative embodiments, the output of models which leverage other time series signals can be combined, for example data from anticipated future sensors such as a pressure sensor added to CGM, or considerations regarding risk for impending hypoglycemia.

To retrieve disparate signals, the method, system and computer readable medium will: (1) convert/standardize the output of each model into a time series of probabilities over a common time interval (e.g., 2.5 minutes or 5 minutes) where the time series of probabilities tracks the probability of the event of interest, in this case a PISA, with different fidelity depending on the relatedness of the signal to PISA. For example, the output of the Random Forest or AdaBoost models is strongly related to PISA, while the output from a pressure sensor may have weaker relationship and the output from a model predicting hypoglycemia may be only an additional consideration intended to focus embodiments on detection of PISA events with higher chances to trigger a hypoglycemia false alarm; and (2) combine the time series using the iterative Bayesian update procedure described.

Imposing a predefined threshold, e.g., 0.75 or 0.9, on the final time series of posterior probabilities enables detection of events with probabilities exceeding the predefined threshold. Tuning of the predefined threshold results may provide a balance between true detection and false positive calls.

In alternative embodiments, other signals, or external information can be available to the detection method. For example, a separate model that tracks the probability for hypoglycemia can be added to make the method more sensitive to PISAs which may trigger false alarms for hypoglycemia. Such a model could be available from other devices, e.g., insulin pump, communicating with the glucose sensor in a closed-loop control application. Thus, an iterative Bayesian update procedure is proposed to combine different signals and considerations in a single output, as long as the signals and considerations are standardized into compatible time series of probabilities for the event of interest. The iterative Bayesian update procedure works as follows for each time interval.

The procedure is initialized using the output of Model 1:

P¹=P₁(Model 1)

This estimate is updated with the output of Model 2:

$${P}^{2} = \frac{{P}^{1} \cdot {P}_{2} ( Model 2 )}{{P}^{1} \cdot {P}_{2} ( Model 2 ) + ( 1 - {P}^{1} ) ( 1 - {P}_{2} ( Model 2 ) )}$$

This estimate can be further updated with the output of another model, Model 3:

$${P}^{3} = \frac{{P}^{2} \cdot {P}_{3} ( Model 3 )}{{P}^{2} \cdot {P}_{3} ( Model 3 ) + ( 1 - {P}^{2} ) ( 1 - {P}_{3} ( Model 3 ) )}$$

This process can continue as long as there are other models with information to be incorporated, with a final posterior probability being realized once information from all models has been incorporated.

It will be understood that modifications to embodiments disclosed herein can be made to meet a particular set of design criteria. For instance, any of the components discussed herein can be any suitable number or type of each to meet a particular objective. Therefore, while certain exemplary embodiments of the system and methods of making and using the same disclosed herein have been discussed and illustrated, it is to be distinctly understood that the disclosure is not limited thereto but can be otherwise variously embodied and practiced within the scope of the following claims.

It will be appreciated that some components, features, and/or configurations can be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein. Additionally, the disclosure of a range of values is a disclosure of every numerical value within that range, including the end points.

What is claimed is:

1. A system for automatically detecting sensor compression in continuous glucose monitoring, the system comprising:
at least one sensor; and
at least one processor in communication with the at least one sensor, the at least one processor executing at least two machine learning models, wherein the at least one processor is programmed or configured to cause the processor to:
receive, from the at least one sensor, measurement data including at least one time series of blood glucose (BG) measurements measured by the at least one sensor;
determine that the at least one time series of BG measurements is a candidate series including a compression artifact using a first machine learning model; and
generate, using a second machine learning model, a signal output indicating that the at least one time series of BG measurements was obtained while the at least one sensor was subject to compression.

2. The system of claim 1, wherein at least one time series of BG measurements includes plural time stamps, each time stamp being associated with a BG measurement.

3. The system of claim 1, wherein the at least one processor, as configured to determine that the at least one time series of BG measurements is a candidate series, is programmed or configured to cause the processor to:
determine that the at least one time series of BG measurements includes a change in BG measurements across plural time stamps, wherein the change in BG measurements exceeds a threshold.

4. The system of claim 1, wherein the at least one processor, as configured to determine that the at least one time series of BG measurements is a candidate series, is programmed or configured to cause the processor to:
determine, using the first machine learning model, that the at least one time series of BG measurements includes a time series sub-sequence having a drop time-window and having a rise time-window associated with the drop time-window, wherein the time series sub-sequence includes a sequence of time stamps corresponding to at least a portion of the drop time-window and at least a portion of the rise time-window.

5. The system of claim 1, wherein the at least one processor is programmed or configured to cause the processor to:
identify one or more features of the at least one time series of BG measurements; and
input the one or more features into the second machine learning model.

6. The system of claim 1, wherein the at least one processor, as configured to generate a signal output, is programmed or configured to cause the processor to:
predict, in real time via outputting an indication, that the at least one sensor is subject to compression while the at least one sensor is obtaining a BG measurement.

7. The system of claim 1, in combination with:
an insulin delivery system in communication with the at least one processor, wherein the at least one processor is programmed or configured to cause the processor to:
transmit the signal output to the insulin delivery system indicating that the at least one sensor is subject to compression, wherein the signal output will cause the insulin delivery system to perform at least one or more of:
initiating insulin delivery, continuing insulin delivery, disabling an alarm, and/or any combination thereof.

8. The system of claim 2, wherein plural time stamps are separated by any one or more of 30 second intervals, 1 minute intervals, 2.5 minute intervals, and/or 5 minute intervals.

9. The system of claim 1, wherein the at least one processor is programmed or configured to cause the processor to:
execute the first machine learning model and the second machine learning model concurrently.

10. The system of claim 1, wherein the at least one processor is programmed or configured to cause the processor to:
identify, with the first machine learning model, the rise time-window associated with the drop time-window as occurring within a range of 15 minutes to 180 minutes later than the drop-time window in the at least one time series.

11. The system of claim 1, wherein the at least one processor, as configured to determine that the at least one time series of BG measurements is a candidate series, is programmed or configured to cause the processor to:
determine a drop time-window within the at least one time series based on a difference between a first BG measurement and a second BG measurement exceeding a drop time-threshold, wherein the drop time-window begins at a first time stamp associated with the first BG measurement and ends at a second time stamp associated with the second BG measurement; and
determine a rise time-window within the at least one time series based on a difference between a third BG measurement and a fourth BG measurement exceeding a rise time-threshold, wherein the rise time-window begins at a third time stamp associated with the third BG measurement and ends at a fourth time stamp associated with the fourth BG measurement.

12. The system of claim 11, wherein the drop time-threshold is 10 mg/dL and the rise time-threshold is 6 mg/dL.

13. A system for automatically detecting onset of sensor compression in continuous glucose monitoring, the system comprising:
at least one sensor; and
at least one processor in communication with the at least one sensor, the at least one processor executing program code for at least one machine learning model, wherein the at least one processor is programmed or configured to cause the processor to:
receive, from the at least one sensor, measurement data including at least one time series of blood glucose (BG) measurements measured by the at least one sensor;
determine that the at least one time series of BG measurements is a candidate series including BG measurements representing onset of sensor compression;
input a time series sub-sequence of at least one time series of BG measurements into at least one machine learning model; and
generate, using at least one machine learning model, a signal output indicating that at least one BG measurement was obtained while the at least one sensor was subject to compression.

14. The system of claim 13, wherein at least one time series of BG measurements includes plural time stamps, each time stamp being associated with a BG measurement.

15. The system of claim 13, wherein the at least one processor, as configured to determine that the at least one time series of BG measurements is a candidate series, is programmed or configured to cause the processor to:
determine that at least one time series of BG measurements includes a drop time-window, wherein the time series sub-sequence includes plural time stamps within the drop time-window.

16. The system of claim 13, wherein the at least one processor, as configured to generate the signal output, is programmed or configured to cause the processor to:
predict, in real time via outputting an indication, that the at least one sensor is subject to compression while the at least one sensor is obtaining a BG measurement, wherein the prediction is based on a probability value representing a probability that a time series sub-sequence includes a BG measurement obtained while the at least one sensor was subject to compression.

17. The system of claim 16, wherein the at least one processor is programmed or configured to cause the processor to:
determine a maximum probability value of plural probability values, the plural probability values being associated with plural time stamps in the time series sub-sequence that are contained within a drop time-window; and
determine the probability that the time series sub-sequence includes a BG measurement obtained while the at least one sensor was subject to compression based on the maximum probability value.

18. The system of claim 13, in combination with:
an insulin delivery system in communication with the at least one processor, wherein the at least one processor is programmed or configured to cause the processor to:
transmit the signal output to the insulin delivery system indicating that the at least one sensor is subject to compression, wherein the signal output will cause the insulin delivery system to perform at least one or more of:
initiating insulin delivery, continuing insulin delivery, disabling an alarm, and/or any combination thereof.

19. The system of claim 13, wherein at least one time series of BG measurements spans 30 minutes of measurement data measured by at least one sensor.

20. The system of claim 13, wherein the at least one processor, as configured to input a time series sub-sequence of the at least one time series of BG measurements into at least one machine learning model, is programmed or configured to cause the processor to:
identify one or more features of the time series sub-sequence of at least one time series of BG measurements; and
input the one or more features into the at least one machine learning model.

21. The system of claim 20, wherein the one or more features include at least one or more of a raw BG measurement, a start BG value at a first time stamp of the drop time-window, an end BG value at a last time stamp of the drop time-window, a difference between the start BG value and the end BG value, a slope of BG values of the drop time-window, a standard deviation of BG values of the drop time-window, a time of day, a temperature value, a comparison value between BG measurements and raw BG measurements, and/or any combination thereof.

22. The system of claim 13, wherein the at least one processor, as configured to determine that at least one time series of BG measurements is a candidate series, is programmed or configured to cause the processor to:
determine a rolling mean for the at least one time series of BG measurements including a smooth BG value associated with each BG measurement and time stamp pair; and
calculate an indicator value for the smooth BG value at each time stamp t, wherein the indicator value is equivalent to a Boolean true where:
BGₜ−BG_{t-lag}>BG threshold
where t is a current time stamp for which an indicator value is determined, BGₜ is the smooth BG value at time stamp t, BG_{t-lag} is a smooth BG value at a previous time stamp, lag is a measure of time such that t-lag represents the previous time stamp, and BG threshold represents a BG threshold value; and
identify a time series sub-sequence in the at least one time series of BG measurements wherein the time series sub-sequence has a set of indicator values, the set of indicator values beginning at a first time stamp and ending at a second time stamp.

23. The system of claim 22, wherein the lag is equivalent to 5 minutes and the BG threshold is equivalent to 10.0 mg/dL.

24. The system of claim 22, wherein the time series sub-sequence spans at least 2.5 minutes in duration of BG measurements.

25. The system of claim 22, wherein a difference between a first smooth BG value associated with the first time stamp and a second smooth BG value associated with the second time stamp is greater than 7.5 mg/dL.

26. A computer-implemented method for generating at least one machine learning model to accurately detect sensor compression in continuous glucose monitoring, the method comprising:
receiving, as an input to a processor, at least one training dataset, the at least one training dataset including plural time series of blood glucose (BG) measurements;
determining plural time series sub-sequences based on the training dataset, wherein at least one time series sub-sequence includes at least one BG measurement value that is less than a compression estimate threshold;
extracting one or more features from each of the plural time series sub-sequence;
inputting the one or more features from the plural time series sub-sequences into at least one machine learning model for training; and
detecting a sensor compression based on providing at least one time series of BG measurements as input to the at least one machine learning model.

27. The computer-implemented method of claim 26, wherein the compression estimate threshold is equivalent to 85 mg/dL.

28. The computer-implemented method of claim 26, comprising:
labelling each of the plural time series sub-sequences with an indication that the time series sub-sequence includes a compression artifact or that the time series sub-sequence does not include a compression artifact.

29. The computer-implemented method of claim 26, comprising:
transmitting a signal output to an insulin delivery system, the signal output indicating detection of sensor compression, wherein the signal output causes the insulin delivery system to perform at least one or more of:
initiating insulin delivery, continuing insulin delivery, disabling an alarm, and/or any combination thereof.
